(19) United States
(12) Patent Application Publication
(10) Pub. No.: US 2025/0099716 A1
(43) Pub. Date: Mar. 27, 2025

(54) GUIDE CATHETER

(71) Applicant: Stallion Catheter, Inc., Happy Valley, OR (US)

(72) Inventors: Rushi Vrajmohan Parikh, Hermosa Beach, CA (US); Yohan Chacko, Graceville (AU); Alan Ching-yuen Yeung, Hillsborough, CA (US); Himanshu Singh, Belmont, CA (US); Jason Pfund, San Francisco, CA (US); Stephanie Whalen, San Francisco, CA (US); Justin Lee, Oakland, CA (US); Bretton M. Swope, San Francisco, CA (US); Eric A. Secemsky, Newton, MA (US)

(21) Appl. No.: 18/290,732

(22) PCT Filed: Jul. 21, 2022
(86) PCT No.: PCT/US2022/074029

§ 371 Date: Jan. 19, 2024

Related U.S. Application Data
(60) Provisional application No. 63/203,415, filed on Jul. 21, 2021.

(51) Int. Cl. A61M 25/01; A61M 39/22

(52) U.S. Cl. CPC A61M 25/0113; A61M 39/22

(57) ABSTRACT

A dynamic catheter system and methods for use of the dynamic catheter system. The system can comprise a guide catheter, a guide extension catheter positioned within the guide catheter and configured to extend from a distal end of the guide catheter, and a valve (e.g., a hemostatic valve) at a proximal end of the guide catheter. The guide catheter and the guide extension catheter can include varying diameters and wall thicknesses so that an inner diameter of the guide extension catheter can be maximized to receive a variety of different tools or equipment. The system can further include a wire control mechanism configured to separate two or more wires. The wire control mechanism can be integral with or configured to be removably coupled to the valve.

### BACKGROUND

Embodiments described herein relate to a novel catheter control system. The catheter control system can include a guide catheter, a telescoping guide extension catheter, and a catheter control center. The catheter control center can include various elements such as an advancement mechanism, a hemostatic valve, and a wire storage compartment. Devices and methods that can be used to significantly improve use of the guide catheter and guide extension catheter without adding significant manufacturing and/or assembly cost. Embodiments of advancement mechanisms and methods can be particularly impactful on any medical procedure utilizing guide catheter and guide extension catheter type devices.

### SUMMARY

In some cases, a catheter control system can comprise a guide catheter comprising a distal end and a proximal end, wherein the distal end is configured to be positioned within an artery, a guide extension catheter positioned within the guide catheter and configured to extend from the distal end of the guide catheter, and a catheter control center at a proximal end of the guide catheter, the catheter control center comprising a guide extension advancement mechanism, wherein the guide extension advancement mechanism is in communication with the guide extension catheter and is configured to move the guide extension catheter within the guide catheter and extend the guide extension catheter from the distal end of the guide catheter; and a valve at a proximal end of the catheter control center configured to allow passage of wires and/or devices between the valve and the guide catheter.

The catheter control system of any preceding paragraphs and/or any of the catheter control system disclosed herein can include one or more of the following features. The guide extension catheter can comprise a distal end that comprises a tube and a proximal end that comprises a guide extension catheter wire. The catheter control center can comprise multiple compartments, wherein the multiple compartments can comprise a guide extension catheter wire compartment configured to store the guide extension catheter wire; and a main valve compartment configured to allow passage of wires and devices between the valve and the guide catheter. The guide extension advancement mechanism can be configured to deliver equipment through the guide extension catheter. The guide catheter can comprise a rail or channel that the guide extension catheter moves along within the guide catheter. The guide extension advancement mechanism can comprise a branched valve comprising a first channel and a second channel, wherein the first channel is configured to deliver equipment and the second channel is configured to allow the guide extension catheter and/or the guide extension catheter wire to pass. The guide extension catheter wire can be in communication with the guide extension catheter and the guide extension catheter moves in coordination with the guide extension catheter wire. The guide extension catheter wire can be in communication with the first channel. The guide extension catheter wire can be in communication with the second channel. The guide extension advancement mechanism can be configured to move the guide extension catheter wire and/or the guide extension catheter within the guide catheter. The guide extension advancement mechanism can comprise a slider mechanism. The guide extension advancement mechanism can comprise a spool mechanism. The guide extension advancement mechanism can comprise a contact wheel mechanism. The guide extension advancement mechanism can comprise a screw mechanism. The guide extension advancement mechanism can comprise a rack and pinion mechanism. The guide extension advancement mechanism can comprise a non-contact mechanism. The guide catheter can be configured to be placed into arteries for cardiological or vascular procedures. A catheter control system can comprise one or more of the features of the foregoing description. A method of using the catheter control system can comprise one or more features of the foregoing description.

Any of the features, components, or details of any of the arrangements or embodiments disclosed in this application, including without limitation any of the guide catheter and guide extension catheter system embodiments disclosed below, are interchangeably combinable with any other features, components, or details of any of the arrangements or embodiments disclosed herein to form new arrangements and embodiments.

### BRIEF DESCRIPTION OF THE DRAWINGS

Various embodiments of the devices and methods of the present disclosure are described herein with reference to the drawings wherein:

FIG. 1 illustrates a guide catheter for use in medical procedures;

FIGS. 2A-2B illustrate a guide catheter shaft with telescoping guide extension catheter system;

FIGS. 3A-5C illustrate a guide catheter shaft with telescoping guide extension catheter system;

FIGS. 6A-6F illustrate a guide catheter with a telescoping guide extension catheter that incorporates a branded wire advancement mechanism;

FIGS. 7A-7D illustrate a wire advancement mechanism using a slider mechanism;

FIGS. 8A-8D illustrate a wire advancement mechanism using a spool mechanism;

FIGS. 9A-9D illustrate a wire advancement mechanism using a contact wheel mechanism;

FIGS. 10A-10B illustrate a wire advancement mechanism using a screw mechanism;

FIGS. 11A-11D illustrate a wire advancement mechanism using a rack and pinion mechanism:

FIGS. 12A-12B illustrate a wire advancement mechanism using a non-contact advancement mechanism:

FIGS. 13-16 illustrate examples of handles and/or grips that can be used with the any of the guide extension advancement mechanisms and/or guide catheters;

FIGS. 17A-22B illustrate examples of a catheter control system with a guide extension advancement mechanism to actuate a guide extension catheter within the guide catheter;

FIG. 23 illustrates example guide catheter shafts with a telescoping guide extension catheter system;

FIG. 24A illustrates a cross-sectional view of an example guide catheter shaft with an example telescoping guide extension catheter system and a side view of a portion of a distal end of the example guide catheter shaft shown in FIG. 23;

FIG. 24B illustrates the distal end of the guide catheter shaft shown in FIG. 24A;

FIG. 24C illustrates a proximal end of the guide catheter shaft shown in FIG. 24A;

FIG. 25A illustrates a cross-sectional view of an example guide catheter shaft with an example telescoping guide extension catheter system and a side view of a portion of a distal end of the example guide catheter shaft shown in FIG. 23;

FIG. 25B illustrates the distal end of the guide catheter shaft shown in FIG. 25A;

FIG. 25C illustrates a proximal end of the guide catheter shaft shown in FIG. 25A;

FIGS. 26-28 illustrate various examples of a failsafe mechanism for a catheter system;

FIGS. 29A-29D illustrate various views of an example spool system;

FIGS. 30A-30C illustrate perspective views of an example anchor system;

FIGS. 31-36B illustrate various examples of storage mechanisms for a catheter wire.

### DETAILED DESCRIPTION

Embodiments described herein relate to a novel catheter control system. The catheter control system can include a guide catheter, a telescoping guide extension catheter, and a catheter control center. The catheter control center can include various elements such as an advancement mechanism, a hemostatic valve, and a wire storage compartment. Devices and methods that can be used to significantly improve use of the guide catheter and guide extension catheter without adding significant manufacturing and/or assembly cost. Embodiments of guide catheter devices and methods can be particularly impactful on accessing vasculature. In addition to coronary vascular procedures, any technology described herein (i.e. novel catheter control system) can be applied to any vascular procedures, including but not limited to neurovascular, renovascular, and other peripheral vascular procedures.

The catheter control system described herein can eliminate the need to place guide extension catheter during the middle of a procedure. The catheter control system can allow for easier movement of the guide extension catheter. The catheter control system can provide for less wire confusion and wire wrap and the extension wire can be neatly contained within catheter control center and/or advancement mechanism. In some cases, the guide extension catheter can be easy to move forward and preserves tactile feedback and the guide extension wire can avoid wrapping with other wires. The catheter control system can allow for simpler hand placement for the practitioner and can enable more control and ease of use for the practitioner compared to existing guide catheter and guide extension catheter products. The catheter control system creates opportunities to improve the design and performance of guide catheters and guide extension catheters. For example, the catheter control system with the built in guide extension catheter and/or advancement mechanism can allow for a guide extension catheter tip that can be as soft or softer than existing guide catheter extensions. In some cases, the upgraded guide extension catheters can have an outer surface that allows for easier movement within the guide catheter and vasculature without adding significant manufacture and/or assembly cost. In another example embodiment, the catheter control system can allow for an increased inner diameter of the guide extension catheter to allow for more space for equipment to pass through. Additionally, the built-in guide extension catheter can allow for easier placement of the guide catheter by creating more stiffness within the guide catheter.

Current guide catheters can be used to make it easier to enter a vessel with other devices or instruments. Guide catheters can be used to facilitate placement of balloons and stents for angioplasty and stenting or other procedures.

Current guide extension catheters, separate catheters that are placed within guide catheters, can be used during a medical procedure. Guide extension catheters can be inserted through the catheter past a hemostatic valve and in coordination with other wires or equipment delivered through the catheter. The guide extension catheter can often help as it provides more support to the guide catheter and can make it easier to deliver equipment such as a stent and/or balloon to a target area. Prior to inserting current guide extension catheters, other equipment being used during the procedure might need to be adjusted or removed. The wire portion of the current guide extension catheters, used to advance or withdraw it, can have a uniform cross-sectional shape and also be sticking out through the hemostatic valve post-insertion. The uniform cross sectional shape of the wire can be rectangular and bulky (flat wire). As a result, current guide extension catheter wires can be in the way when handling other wires within the guide catheter and challenging to insert during a procedure.

The procedure for delivering equipment for a percutaneous coronary intervention can include various steps. The radial artery or femoral artery can be accessed and a sheath can be placed. A diagnostic angiogram can be performed using a diagnostic catheter showing a lesion in the particular territory (e.g. mid right coronary artery [RCA]). The diagnostic catheter can be removed and the operator can prepare for percutaneous coronary intervention. A Tuohy-Borst valve or similar hemostatic valve device can be connected to the back of the guide catheter; a manifold is then connected to the hemostatic valve and the guide catheter is flushed. A standard J-tipped guide wire (typically 0.035 to 0.038 inch diameter) can then be fed into the hemostatic valve and guide catheter. The hemostatic valve can be opened slightly to allow the wire to slide in. A wire can be advanced in the vessel to the ascending aorta following with the guide catheter. Once the guide catheter is near the aortic root the wire can be removed and the operator can aspirate and flush the catheter.

The right coronary artery ostium can be engaged with the guide catheter. A standard 0.014 inch coronary guidewire can be advanced into the hemostatic valve, into the guide catheter, and advanced beyond the mid RCA lesion to the distal vessel. If the operator is able to, a compliant balloon can be advanced over the coronary guidewire to the lesion and inflated to pre-dilate the lesion. Then the balloon can be removed and the operator can assess that the balloon has adequately expanded the lesion. If able to, the operator can advance a stent to the lesion. However, in some cases, the operator may not be able to deliver equipment due to the calcified and/or tortuous nature of the lesion/vessel or lack of adequate guide catheter support.

At this point, the operator can insert a guide extension catheter for additional support after first removing the stent (or balloon). After inserting the guide extension catheter over the coronary guidewire, the stent or balloon can then be re-advanced over the coronary guidewire. It is recommended, with existing guide extension catheter devices, the operator slide the guide extension catheter over the shaft of a balloon or stent delivery system in the coronary artery to provide more of a rail and reduce risk of injuring the proximal vessel. In some circumstances, the operator may determine that it is not ideal to have to insert a guide extension catheter as a separate device due to the need to remove the balloon/stent, having multiple wires exiting the hemostatic valve, and cost considerations. Therefore, the operator may try several alternative techniques to avoid the use of a guide extension catheter. The decision can be based on operator comfort and experience, time, and cost. As described above, the catheter control system described herein can be helpful to allow for a single device that can provide the guide catheter, guide extension catheter, and/or an advancement mechanism that can provide control of the catheter control system and routing of wires and devices within the catheter control system.

### Catheter Control System

It can be beneficial to have a catheter control system that utilizes a guide catheter, a telescoping guide extension catheter, and a catheter control center. The catheter control center can include an integrated control center with an advancement mechanism, wire storing and/or holding device, and an integrated hemostatic valve.

It can be beneficial to have a guide catheter that utilizes a built in guide extension catheter that can allow for easy deployment of the guide extension catheter when needed. In such cases, the procedure would follow the steps above but if the operator is not able to deliver equipment due to the calcified/tortuous nature of the lesion/vessel, then the operator can utilize the built in guide extension catheter for additional support. In such cases, the guide extension catheter portion of the device can be advanced over the coronary guide wire and balloon or stent. Then the balloon or stent can be advanced to the lesion. The guide extension catheter portion can often help as it provides more support from the guide catheter and makes it easier to deliver equipment. In traditional cases, if the stent does not cross the lesion, the operator may have to remove the stent, re-advance a non-compliant or compliant balloon and re-balloon. With the built in guide extension catheter device as described in more detail below, the operator can still have the options of a buddy wire or wiggle wire. In other cases, the operator can take the balloon (compliant) past the lesion and inflate at low pressure (approximately 4 atm) to anchor the guide catheter. Then the operator can slide the guide extension catheter past the lesion, remove the balloon, and advance the stent. Then the operator can un-sheath the stent in the lesion.

The built in guide extension catheter can provide extra support without needing additional equipment (i.e. a separate guide extension catheter). Additionally, the built in guide extension catheter can allow for a wire advancing mechanism that does not go through the hemostatic valve so it can be easier to identify, manipulate, and keep separate from the coronary wire and/or balloon or stent wires. The built in guide extension catheter can have design differences that can allow for less trauma to the proximal vessel and more support, for example, in the subclavian region. Additionally, the built in guide extension catheter can save steps during a procedure and can save time. In some cases, the built in guide extension catheter device can allow for easy guide catheter engagement as opposed to using more challenging guide catheters for coronary engagement. For example, for the RCA, the operator can opt to use a Judkins Right 4 (JR4) guide catheter rather with a built in guide extension catheter than an Amplatz Left 0.75 guide catheter, which provides more support than the JR4 but is harder to engage and has a higher risk of proximal vessel dissection. This can be important in an acute, time-critical setting. It can also be useful for engaging coronary arteries post transcatheter aortic valve implantation, which can be challenging with currently available guide catheters.

The catheter control system device described herein can include a guide catheter to be placed into human arteries for vascular (including but not exclusive to coronary artery, peripheral vascular or neurovascular) procedures, to act as a guide for more effective support and delivery of devices such as stents and balloons through the guide catheter and into the artery (for example, within the coronary arteries). The catheter control system can include two key elements, a guide catheter design that contains within it a plastic tube or a guide extension catheter that is able to telescope out and in of the distal end of the guide catheter. The guide catheter can include a telescoping feature for the guide extension catheter. In some cases, the guide catheter with a built in guide extension catheter can be used for a percutaneous coronary intervention or a coronary angioplasty. The catheter control system can include a catheter control center that is integral with the guide catheter and can house an advancement mechanism including the guide catheter wire and a hemostatic valve as described in more detail below.

### Guide Catheter

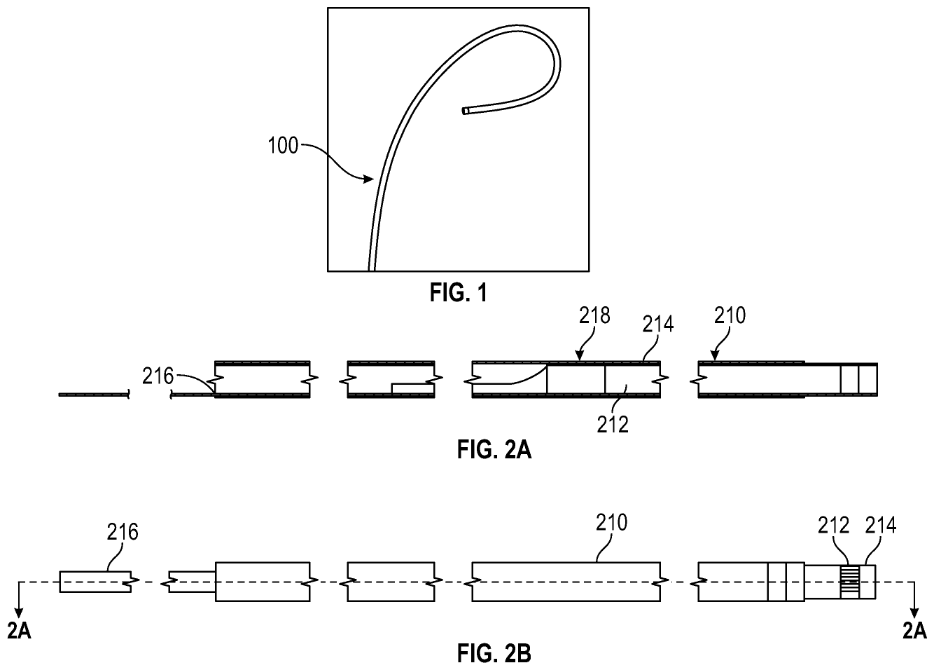

A standard guide catheter 100 is depicted in FIG. 1. A guide catheter can be a round and hollow plastic tube. The plastic can be braided with metal for added stability. The hollow tube can be inserted percutaneously into arteries, for example, through the wrist or groin, and can be navigated to an opening of a coronary artery where the distal end of the guide catheter and/or guide extension catheter can sit. Hence, the guide catheter and/or guide extension catheter can be very small in diameter, ranging from 0.05-0.10 inches (about 0.05-0.10 inches) in diameter. The guide catheter can be used as a conduit or protective placeholder to facilitate delivery of other materials into the coronary artery. For example, a thin metal wire (or coronary wire) can be placed in the middle of the guide catheter over which one or more stents or balloons can be passed within the inner diameter of the guide catheter.

### Guide Extension Catheter

Current guide extension catheter designs can be inserted into a guide catheter after the guide catheter has already been seated into the coronary artery. The distal section of the guide extension catheter can be pushed beyond the distal end of the guide catheter into tortuous and/or heavily calcified arteries to extend the support offered by the guide catheter by providing additional support for delivery of balloons/stents. However, as described herein, the use of a separate guide extension catheter device can create additional complications and difficulties throughout the medical procedure. Therefore, a separate guide extension catheter device that is inserted during the procedure may not be ideal.

The guide catheter and telescoping guide extension catheter described herein can incorporate the guide extension catheter into the guide catheter device.

Current guide extension catheters used with guide catheters must travel through a hemostatic valve and through the guide catheter. In contrast, an integrated guide extension catheter is built within the guide catheter and thus does not need to travel through the hemostatic valve.

The guide extension catheter can have a distal end and a proximal end. The guide extension catheter can be arranged to extend from the distal end of the guide catheter into the necessary vasculature, for example, the arteries. The proximal end of the guide extension catheter can be in communication with the proximal end of the guide catheter and/or any actuation device that can be used to move or actuate the guide extension catheter.

FIG. 2A illustrates a cross section of an embodiment of a guide catheter shaft 210 with a telescoping guide extension catheter 212 arranged in a proximal to distal arrangement. As illustrated in FIG. 2A, the guide extension catheter 212 can include a cylindrical portion 214 and a wire portion 216. The wire portion 216 is on the proximal portion of the guide extension catheter. The wire portion 216 can allow for control and manipulation of the guide extension catheter. In some cases, the wire portion can be a flat wire. The guide extension catheter 212 can be incorporated into the guide catheter 210 to allow for the guide extension catheter to move in a proximal to distal and a distal to proximal direction within the inner diameter of the guide catheter.

As illustrated in FIG. 2A, the guide extension catheter 212 can have a proximal portion 216 that has a smaller diameter and a distal portion 214 that has a larger inner and outer diameter than current guide extension catheters. In some cases, the guide catheter 210) and the distal portion 214 of the guide extension catheter 212 can be concentric. In some cases, the outer diameter of the distal portion 214 of the guide extension catheter 212 can be sized to fit within the inner diameter of the guide catheter 210. In some cases, the outer diameter of the distal portion 214 of the guide extension catheter 212 can be sized to be small enough to allow movement of the guide extension catheter 212 within the guide catheter but can be large enough to allow the inner diameter of the guide extension catheter to allow delivery of instruments or other devices. The outer diameter of the distal portion 214 of the guide extension catheter 212 only needs to be less than the inner diameter of the guide catheter. For example, in some cases, the guide catheter can have an inner diameter of about 2 cm. In some cases, the inner diameter of the distal portion 214 of the guide extension catheter 212 can be about 2 cm or less.

The catheter control system can integrate the guide catheter and guide extension catheter devices. This pre-procedure integration can allow the guide catheter and guide catheter extension designs to be improved. For example, the guide extension catheter within the catheter control system can have the largest possible inner diameter, given the guide extension catheter does not have to be passed from the proximal most to distal most portion of the guide catheter after the guide catheter has already been placed within the patient's body. In some cases, the guide catheter and guide extension catheter can have a variety of sizes and the inner diameter of the guide extension catheter can be similar to the inner diameter of an equivalently sized guide catheter.

In some cases, given the guide catheter and guide extension catheter are pre-loaded together, both can be made with thinner walls to achieve the same guide catheter behavior. The result is a larger inner diameter within the guide extension catheter, which results in additional space for practitioners, relative to current guide extension catheters. Smaller wall thickness can be achieved by various means, such as using smaller braiding patterns or using the latest materials tailored to thin walled catheters (e.g., teflon liners, thermoplastic outer extrusions, and high tensile wires). The guide extension catheter 212 can have a transitional portion 218 between the proximal portion 216 and the distal portion 214. The transitional portion 218 can transition from the smaller diameter wire of the proximal portion 216 to the larger diameter distal portion 214. In some cases, the inner wall of the distal portion 214 of the guide extension catheter 212 can be thinner than traditional guide extension catheter devices allowing the guide extension catheter 212 to be softer.

FIG. 2B illustrates an embodiment of a guide catheter shaft 210 with some portions of the guide catheter cut out to show the guide extension catheter 212 within the guide catheter 210. FIG. 2A illustrates a horizontal cross section through line A-A shown in FIG. 2B.

In some cases, the distal portion of the guide extension catheter could be designed to be formed from a softer material or re-shaped to promote functionality. For example, the distal tip of the guide extension catheter can be formed from materials such as a thermoplastic nylons and Pebax. In some cases, the guide extension catheter can be formed from Polytetrafluoroethylene (PTFE). In some cases, the guide extension catheter can have a hydrophilic coating to aid deliverability. In some cases, the transitional portion 218 of the cylindrical part of the guide extension catheter could be stiffer (more tightly braided) to promote control/movement (couple with the softer distal portion). In some cases, the guide extension catheter can be a coil-reinforced device that provides flexibility and kink resistance during delivery through vessels.

In some cases, the guide catheter can include a rail or channel that the guide extension catheter can move along within the guide catheter. The rail or channel can provide a path for the guide extension catheter to move along to prevent any twisting or tangling of a guide extension wire and/or the guide extension catheter while it moves within the guide catheter.

In some cases, the mother/child design of the guide catheter and guide extension catheter system can provide additional benefits. Ease of placement and position maintenance of the guide catheter can be improved. For example, the overall stiffness of the mother/child combination can be higher than just a guide catheter alone. In another embodiment, adding stiffness to the guide catheter could provide key support. The portion of the guide catheter that traverses the subclavian artery can have extra braiding creating a stiffer component. In some cases, preloading or integrating of the guide extension catheter can change the properties of the guide catheter as it enters the aorta and cardiac anatomy. When the guide extension catheter is built into the guide catheter, the distal end of the guide extension catheter may be able to be softer than traditional guide extension catheters as it does not have to go over a wire, through the hemostatic valve, and/or up the aorta. However, the guide extension catheter and the distal tip of the guide extension catheter may still need to maintain some similar level of thickness to provide support.

In some cases, the device can provide tactile feedback that allows the operator to feel the guide extension catheter movement and pressure feedback. The tactile feedback can be important and allow for ease of use by the operator. In some cases, a wire based mechanism on the proximal portion 216 of the guide extension catheter can be used to provide this tactile feedback. Actuator mechanisms can also be used that allow for similar feedback. For example, the advancement mechanisms described herein (for example the sliding knob mechanism described in detail below) for sliding or moving the guide extension catheter forward can provide the same desirable tactile feedback operators are accustomed to with atherectomy devices (rotational and orbital atherectomy) and provide a familiar user experience for the operator. In some embodiments, in lieu of or in addition to an advancement mechanism within the catheter control center, the guide extension catheter proximal portion wire itself can be adjusted to allow for greater control and tactile feedback.

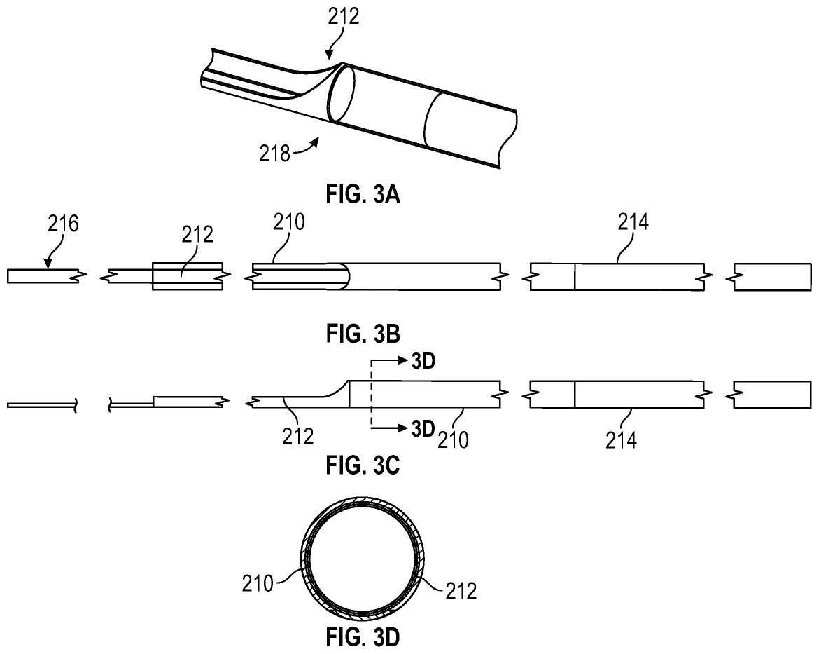

FIGS. 3A-5C illustrate a guide catheter and a guide extension catheter. As shown in FIGS. 3A-3D the guide extension catheter 212 can include a proximal portion 216 including a guide extension wire and a distal portion 214 with a cylindrical braided section. FIG. 3A illustrates a view of the transitional portion 218 of the guide extension catheter 212 that allows a transitional feature between the proximal portion 216 including the guide extension wire and the distal portion 214 with the cylindrical braided section. FIGS. 3B-3C illustrates a view of the guide extension catheter 212. FIG. 3D illustrates a cross section for the guide catheter 210 and guide extension catheter 212 that is a cross section of line 3D-3D in FIG. 3C. FIG. 3D illustrates the concentric nature of the guide extension catheter 212 within the guide catheter 210. As shown in FIG. 3D, the inner diameter of the guide catheter is sized to fit an outer diameter of the guide extension catheter that is as close as possible to the inner diameter of the guide catheter. This arrangement can allow for a close fitting concentric arrangement as shown in FIG. 3D while still allowing for the guide extension catheter to be advanced within the guide catheter without resistance.

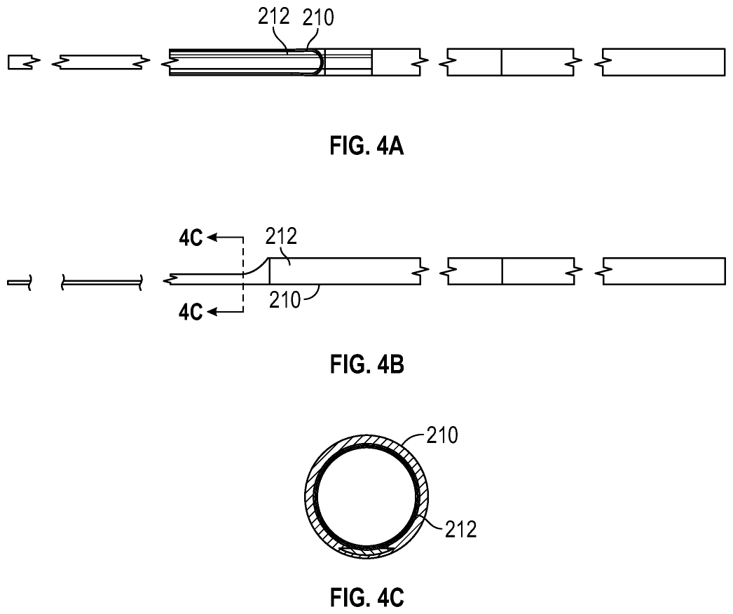

FIGS. 4A-4C illustrate a guide catheter 210 and a concentric guide extension catheter 212 device. FIG. 4C illustrates a cross section for the guide catheter 210 and guide extension catheter 212 that is a cross section of line 4C-4C in FIG. 4B.

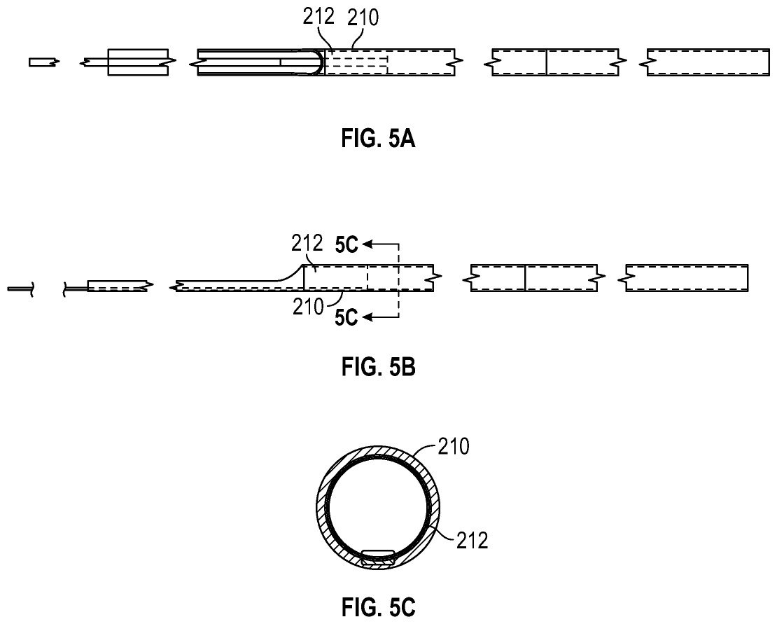

FIGS. 5A-5C illustrated an embodiment of the guide extension catheter with a flat wire. FIG. 5C illustrates a cross section for the guide catheter 210 and guide extension catheter 212 that is a cross section of line 5C-5C in FIG. 5B.

### Catheter Control Center

As described herein, the catheter control system can include a catheter control center which can incorporate an advancement mechanism, a guide extension catheter wire, and/or a hemostatic valve. In some cases, the catheter control center can incorporate one or more of these components within a housing or other enclosure providing a user friendly device that can be controlled and manipulated by the operator. The distal end of the catheter control center can be attached to the proximal end of the guide catheter and the guide extension catheter and guide extension catheter wire can move in a proximal to distal or distal to proximal direction within both the guide catheter and the catheter control center.

### Advancement Mechanisms

The catheter control system can incorporate an actuator for advancing and/or retracting the telescoping guide extension catheter. The actuator at the proximal end of the guide catheter can incorporate various actuation features. The actuator can be incorporated within a catheter control center. In some embodiments, the catheter control center can incorporate an advancement mechanism that uses an actuation device or mechanism that can provide distal and proximal movement, preserve tactile feedback, prevent wire wrapping, have little change to existing components, and allow simple manufacturing and setup.

In some cases, the valve in communication with the advancement mechanism for extending or actuating the telescoping guide extension catheter within the guide catheter and the hemostatic valve are not incorporated into the same housing. In these cases, the valve can be positioned as a pre-hemostatic valve or a post-hemostatic valve depending on the positioning of the hemostatic valve in relation to the positioning of the advancement mechanism. The pre-hemostatic valve can include a device with a secondary valve or branched valve positioned proximal to the hemostatic valve. This configuration can incorporate the guide catheter, guide extension catheter, and advancement mechanism into one piece and allow for no interruptions and encourage use of the guide extension catheter which can make it easier to use. With a branched valve configuration there could be no seal required in the mechanism, however, in some cases, it can require the addition of a second valve adjustment process because the two valves are in two locations. In other cases, the pre-hemostatic valve can include an enclosed mechanism enclosed within the guide catheter device and positioned proximal to the hemostatic valve. The enclosed mechanism may not require an independent open/close mechanism of the valve to advance the guide extension catheter. In some cases, the enclosed mechanism can have diminished tactile feedback.

In some cases, the guide catheter can incorporate the telescoping guide extension catheter using a valve that is incorporated distal to the hemostatic valve or post-hemostatic valve. The post-hemostatic valve may not require a seal. Additionally, the post-hemostatic valve can be formed from two pieces, may require setup assembly, and/or may impede wire manipulation area for other devices.

In some cases, the guide extension catheter wire can have multiple cross-sectional shapes and sizes. For example, the distal portion of the wire could have a rectangular cross section (i.e., flat wire) and the proximal portion of the wire could have a circular cross section (i.e., round wire). The proximal portion of the wire is near or within the catheter control center. Customizing the proximal portion of the wire maximizes the catheter control center's ability to store the wire, actuate the guide extension catheter, and optimize feedback for the practitioner. Customizing the distal portion of the wire allows for optimal wire bending characteristics within the guide catheter, which impacts advancement and retraction behavior of the guide extension catheter.

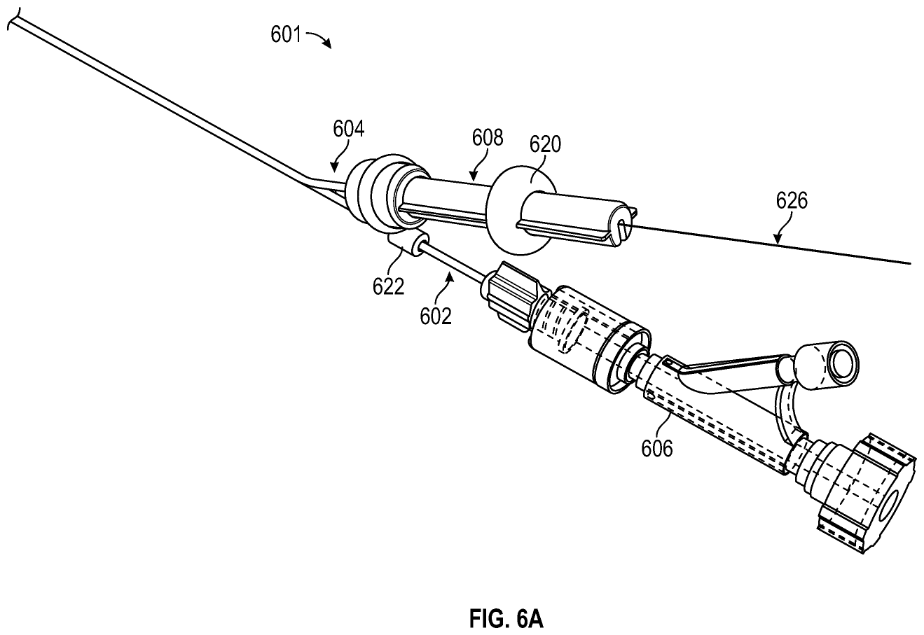

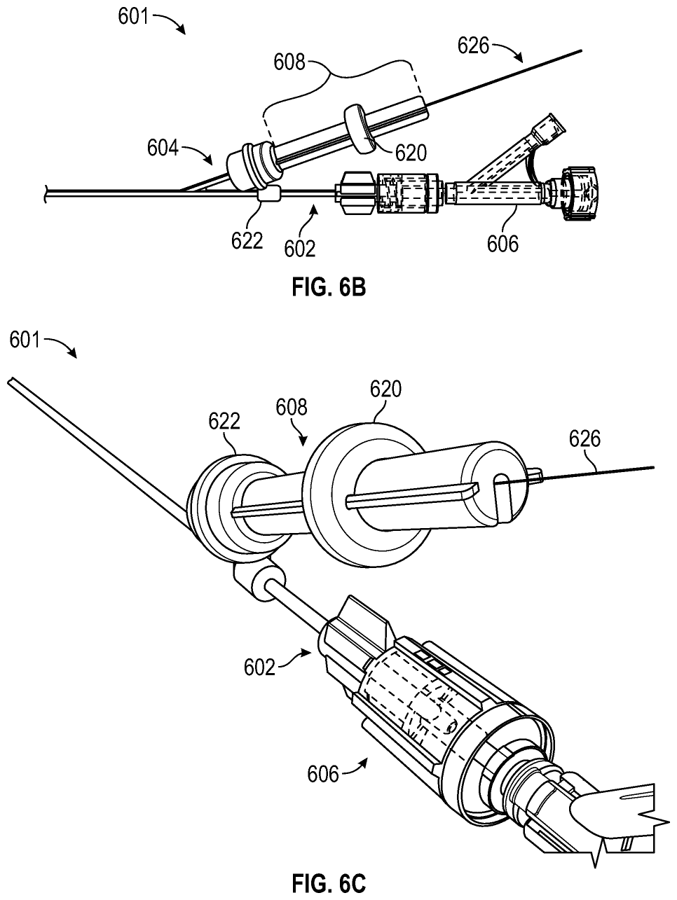

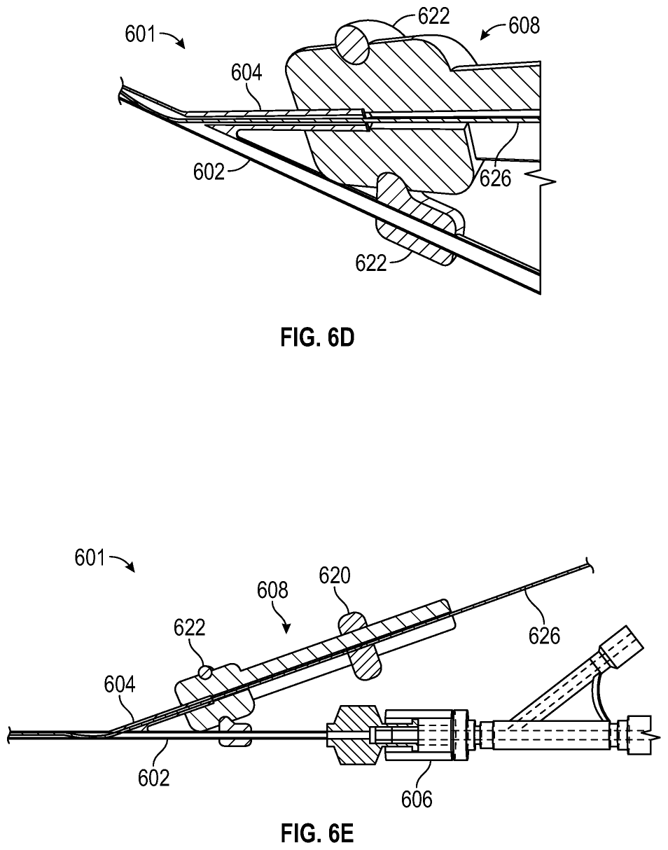

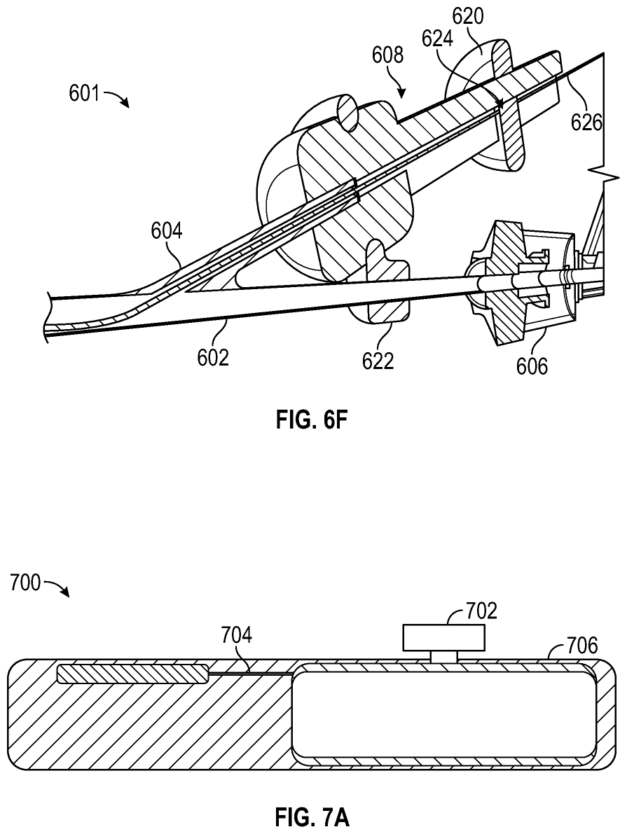

FIGS. 6A-6E illustrate an embodiment of a guide catheter with a telescoping guide extension catheter that incorporates a guide extension advancement mechanism. In some cases, the guide extension advancement mechanism can be a branched wire advancement mechanism which can include a branched slider.

FIG. 6B illustrates a branched device 601 with a 20 degree branch. The first branch 602 of the branched device can include a hemostasis valve 606. The second branch 604 can include a guide extension advancement mechanism 608. For example, as illustrated in FIG. 6A-6F, a guide extension advancement mechanism 608 can be a slider mechanism 620 that can actuate the guide extension catheter. In some cases, the slider mechanism 620 can have a 5-10 cm (about 5-10 cm) distance of slider travel per single slider movement. In some cases, the full movement of the slider (over one or more slider movements) can be 5 cm, 10 cm. 15 cm, 20 cm, 25 cm, 30 cm, 35 cm. 40 cm, 45 cm, 50 cm, 55 cm, 60 cm, 65 cm, 70 cm, or more (about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, or more). In some cases, the full movement of the slider (over one or more slider movements) can be between about 5 cm and about 70 cm, about 10 cm and about 65 cm, about 15 cm and about 60 cm, about 20 cm and about 55 cm, about 25 cm and about 50 cm, about 30 cm and about 45 cm, or about 35 cm and about 40 cm. In some cases, the full movement of the slider (over one or more slider movements) can be at least 25 cm (about at least 25 cm) or more. In some cases, the slider mechanism 620 can have a 15.5 mm diameter or can be any diameter that is comfortable for the operator to hold and/or manipulate. FIG. 6C shows a wire 626 for manual pushing or longer advancement. FIGS. 6D-6F illustrate a cross section of the branched wire guide catheter device 601. The branched wire guide catheter device 601 can have a silicone retaining ring 622 for added support between the first and second branches of the device. In some cases, the slider mechanism 620 can include a wire clamp 624 as shown in FIG. 6F.

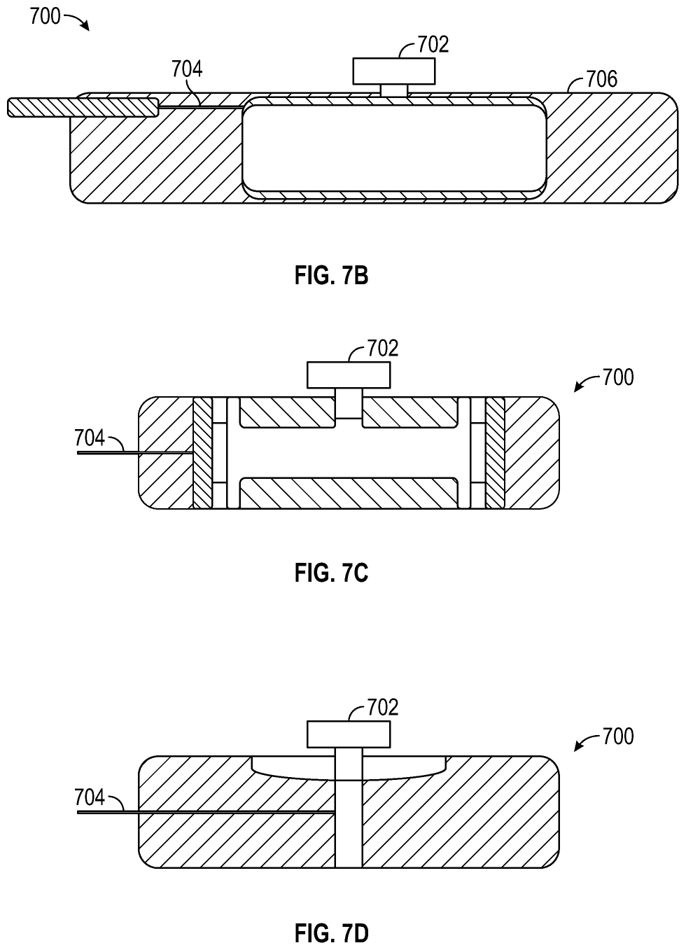

FIGS. 7A-7B illustrates an embodiment of a guide extension advancement mechanism 700 using a slider mechanism 702. The slider mechanism 702 can include an actuator that can be moved along a horizontal axis that runs from a proximal to distal direction of the slider mechanism 702. The slider mechanism 702 can be attached to a wire 704 to actuate the wire 704 within the guide catheter. As the slider mechanism 702 is moved along the horizontal axis, the wire 704 is moved in a distal to proximal and proximal to distal direction parallel to the horizontal axis. FIG. 7A illustrates a first position of the slider mechanism 702 with the wire retracted into the mechanism in a proximal most direction. FIG. 7B illustrates a second position of the slider mechanism 702 with the wire 704 extended to a position distal to the first position and the guide extension catheter can be extended in a distal direction. The slider mechanism can include a casing 706 that forms an enclosed structure surrounding the wire 704. The slide mechanism 702 can provide tactile feedback for the operator. The guide extension advancement mechanism 700 may require a specific grip that can use a thumb to move the slider mechanism 702. In some cases, if the slider mechanism 702 needs to be moved past the thumb length the operator may need to readjust the grip on the device.

FIGS. 7C-7D illustrate guide extension advancement mechanisms that can be used. FIG. 7C illustrates an o-ring sliding cylinders that can be used within the guide extension advancement 700. The o-ring sliding cylinders of the guide extension advancement 700 can include a slider mechanism 702 and a wire 704 that can be moved in the proximal to distal or distal to proximal direction by the movement of the slider mechanism 702. FIG. 7D illustrates another example of a guide extension advancement mechanism 700 with a slider mechanism 702 that can be used and would require no seal. The guide extension advancement mechanism 700 can include a slider mechanism 702 and a wire 704 that can be moved in the proximal to distal or distal to proximal direction by the movement of the slider mechanism 702.

The guide extension advancement mechanism 700 using the slider mechanism 702 of FIGS. 7A-7D can be used with the system described with reference to FIGS. 6A-6F and can be used in place of the guide extension advancement mechanism 608 in FIGS. 6A-6F.

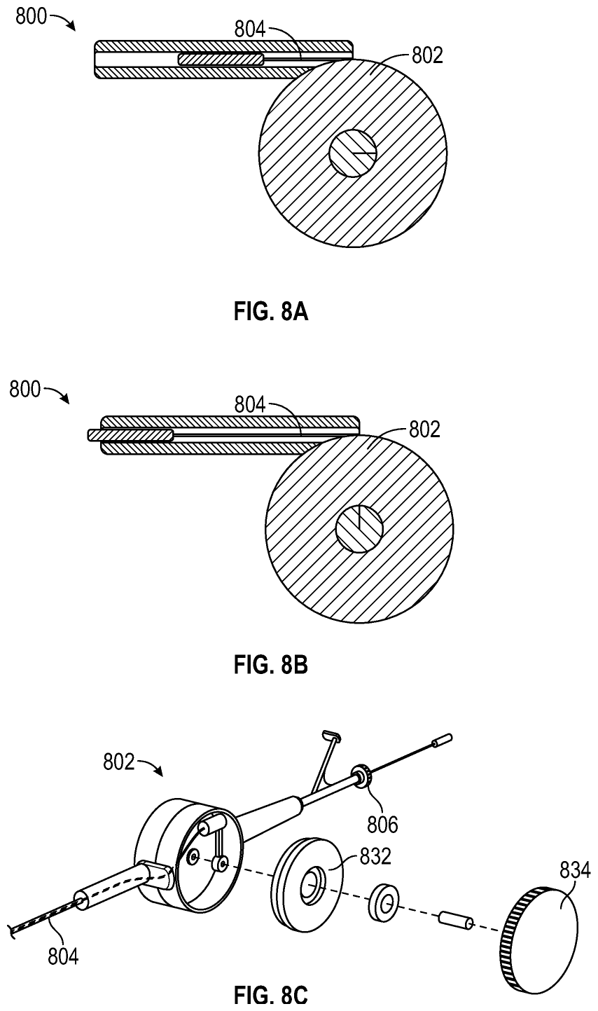

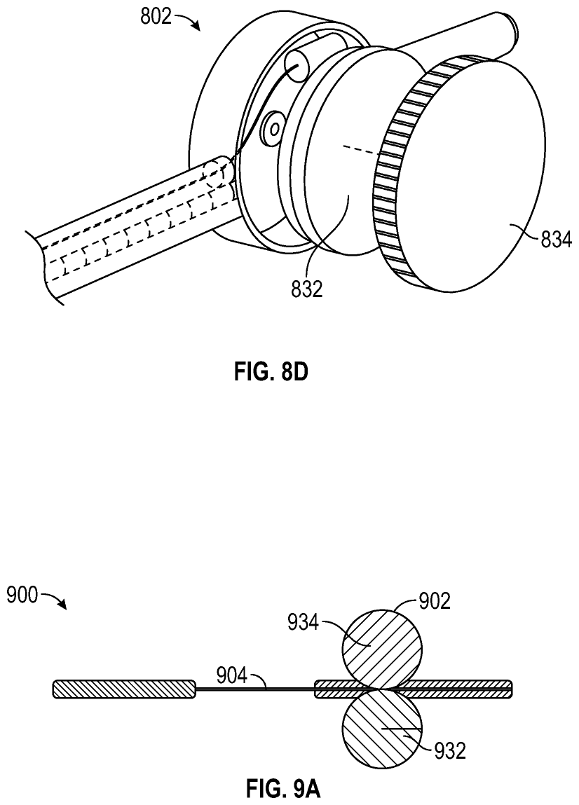

FIGS. 8A-8B illustrate an embodiment of a guide extension advancement mechanism 800 using a spool mechanism 802. The guide extension advancement mechanism 800 with the spool mechanism 802 can be actuated with a one-handed fixed grip. The wire 804 connected to the guide extension catheter can be wrapped around the spool and the spool can be actuated to move the guide extension catheter from a proximal to distal and a distal to proximal configuration. FIG. 8A illustrates a first position of the spool mechanism 802 with the wire 804 retracted into the mechanism in a proximal most direction. FIG. 8B illustrates a second position of the spool mechanism 802 with the wire 804 extended at a position distal to the first position. The guide extension advancement mechanism 800 with the spool mechanism 802 can provide a compact length since the wire is wrapped around the spool 806 instead of extending from the proximal end of the guide extension advancement mechanism.

FIGS. 8C-8D illustrate an exploded view of a guide extension advancement mechanism with a spool mechanism 802. FIG. 8C illustrates the components of the spool mechanism 802 on the guide catheter device in a distal position to the hemostatic valve 806. The wire 804 can be wound around a wheel 832 and a cap 834 with grooves that can be used to move the wheel 832 and therefore actuate the wire 804. The features of the spool mechanism can include a seal at the distal end to prevent fluid from entering the spool mechanism.

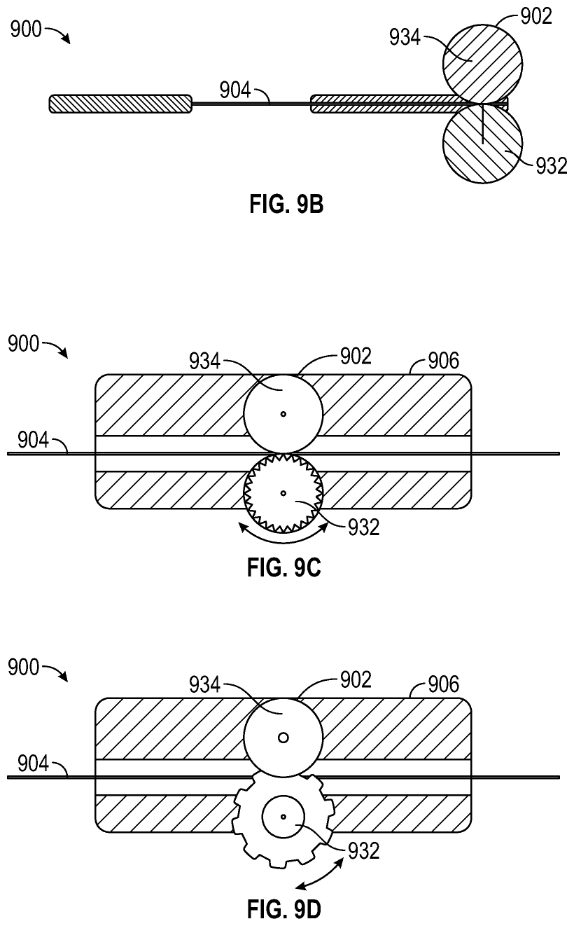

FIGS. 9A-9B illustrate an embodiment of a guide extension advancement mechanism 900 using a contact wheel mechanism 902. The contact wheel mechanism 902 can include two wheels 932, 934 and one or both wheels can move to allow the movement of the wire 904 and the guide extension catheter at the distal end of the wire. The contact wheel mechanism 902 can be more compact than other devices and can allow for a one-handed user friendly fixed grip that can simplify operation for the operator. The wire is arranged to move within the two wheels in a proximal to distal and distal to proximal direction which can move the guide extension catheter within the guide catheter and/or arteries. In some cases, an area of the wire that is positioned to move between the wheels can be thicker than the remainder of the wire or can be formed from a material with additional grip to allow for the wire to better contact the wheels. FIG. 9A illustrates a first position of the contact wheel mechanism 902 with the wire 904 in a first position. FIG. 9B illustrates a second position of the contact wheel mechanism 902 with the wire 904 extended at a position distal to the first position.

FIGS. 9C and 9D illustrate other examples of a guide extension advancement mechanism 900 with a contact wheel mechanism 902. The contact wheel mechanisms 902 in FIGS. 9C-9D are similar to the contact wheel mechanism 902 in FIGS. 9A-9B. However, the contact wheel mechanisms 902 is enclosed within a housing 906. The first wheel 934 can be positioned within the housing and the second wheel 932 can be partially positioned within the housing 906. As shown in FIGS. 9C and 9D the second wheel 932 can rotate and can be used to move the wire 904 within the contact wheel mechanism 902.

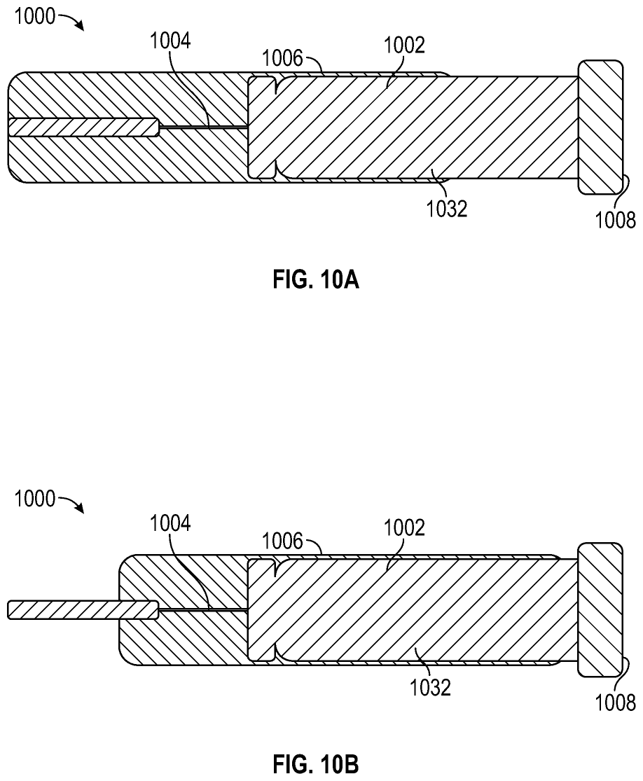

FIGS. 10A-10B illustrate an embodiment of a guide extension advancement mechanism 1000 using a screw mechanism 1002. The screw mechanism can be in communication with a housing 1006 and a wire 1004 that can be attached to the guide extension catheter to move the guide extension catheter within the guide catheter. The screw mechanism 1002 can be designed with a screw-like device 1032 with threads that can move along complementary threads in the inner diameter of the housing 1006. As the screw 1032 is moved from the proximal to distal or distal to proximal direction the wire and guide extension catheter is also moved from the proximal to distal or distal to proximal direction. FIG. 10A illustrates a first position of the screw mechanism 1002 with the wire 1004 in a first position. FIG. 10B illustrates a second position of the screw mechanism 1002 with the wire 1004 extended at a position distal to the first position. The guide extension advancement mechanism 1000 using the screw mechanism 1002 can allow for a fixed grip as the operator can twist the proximal end 1008 of the screw 1032 to move the screw within the housing and thereby move the wire 1004 and guide extension catheter. In some cases, the wire can be attached to points in the housing or run along a guide or rail to prevent the wire from twisting when the screw is rotated.

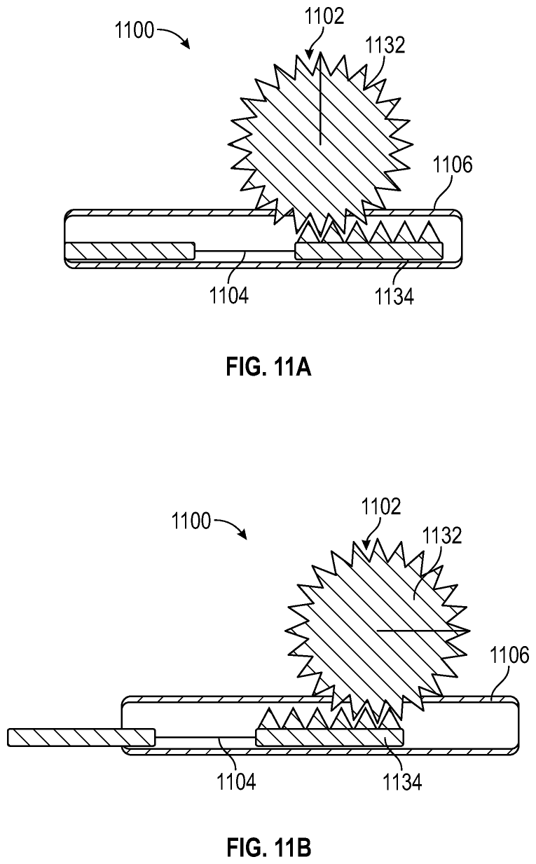

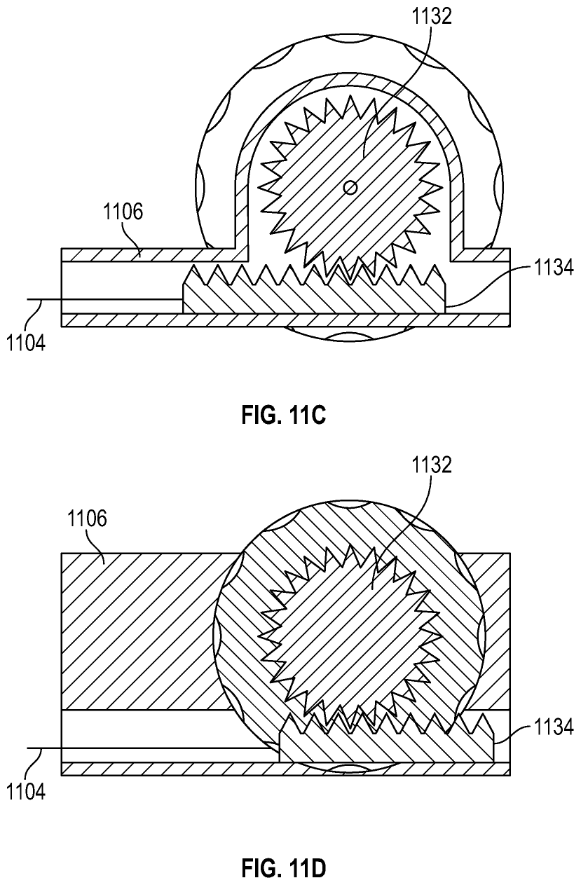

FIGS. 11A-11B illustrate an embodiment of a guide extension advancement mechanism 1100 using a rack and pinion mechanism 1102. The rack and pinion mechanism 1102 can be used to move a wire 1104 and attached guide extension catheter within the guide catheter. The rack and pinion mechanism 1102 can include a circular gear 1132 (pinion) that engages with a linear gear 1134 (rack) which can operate to translate the rotational motion of the circular gear 1132 into linear motion. The operator can move the circular gear 1132. The rotation of the circular gear 1132 can move the linear gear 1134 in a proximal to distal or distal to proximal direction. The movement of the linear gear 1134 can then move the wire 1104 and thereby move the guide extension catheter in a proximal to distal or distal to proximal direction. FIG. 11A illustrates a first position of the rack and pinion mechanism 1102 with the wire 1104 in a first position. FIG. 11B illustrates a second position of the rack and pinion mechanism 1102 with the wire 1104 extended at a position distal to the first position. FIGS. 11C-11D illustrate other examples of guide extension advancement mechanisms 1100 using a rack and pinion mechanism 1102. FIG. 11C illustrates a rack and pinion mechanism 1102 with a finger wheel which can allow the operator to move the circular gear 1132 with their finger. FIG. 11D illustrates a rack and pinion mechanism 1102 with a thumb wheel which can allow the operator to move the circular gear 1132 with their finger.

As shown in FIGS. 11A-11D, at least a portion of the linear gear 1134 can be positioned within a housing 1106 and the circular gear 1132 can extend from the housing 1106. The operator can rotate the circular gear 1132 that extends from the housing 1106 and thereby move the linear gear 1132 and the wire/guide extension catheter in the proximal to distal or distal to proximal direction. The rack and pinion mechanism 1102 can use a well constrained channel to prevent the wire from buckling. The rack and pinion mechanism 1102 can be a fixed grip device that can be actuated with a finger or thumb of the operator.

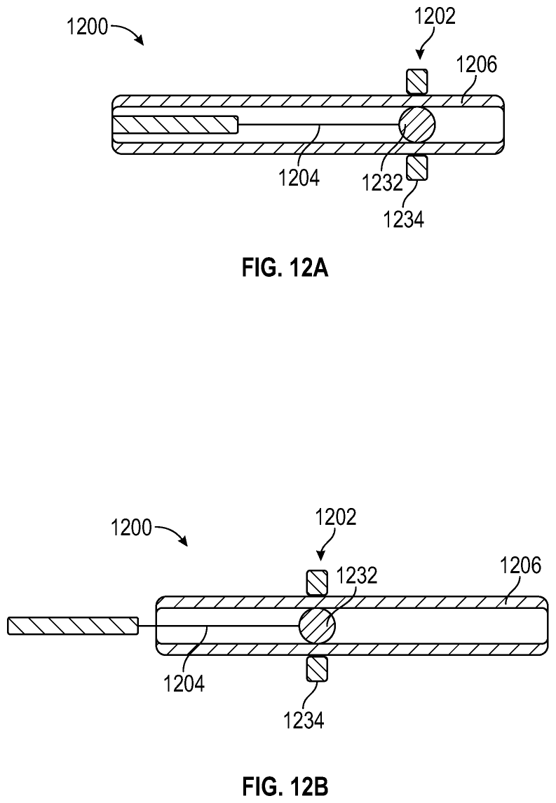

FIGS. 12A-12B illustrate an embodiment of a guide extension advancement mechanism 1200 using a non-contact advancement mechanism 1202. In some cases, the non-contact advancement mechanism can use magnets. A non-contact advancement mechanism 1202 can allow for the guide extension advancement mechanism 1200 to be actuated with a closed system where the housing 1206 encloses the inner component 1232 and the wire 1204 while the outer component 1234 can be positioned outside the housing 1206. FIG. 12A illustrates a first position of the non-contact advancement mechanism 1202 with the wire 1204 in a first position. FIG. 12B illustrates a second position of the non-contact advancement mechanism 1202 with the wire 1204 extended at a position distal to the first position.

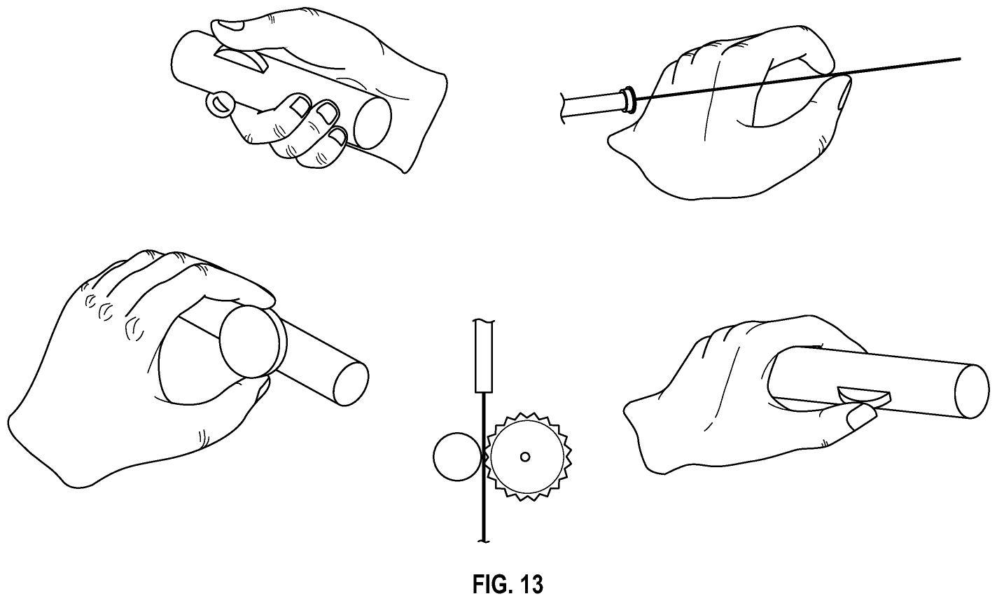

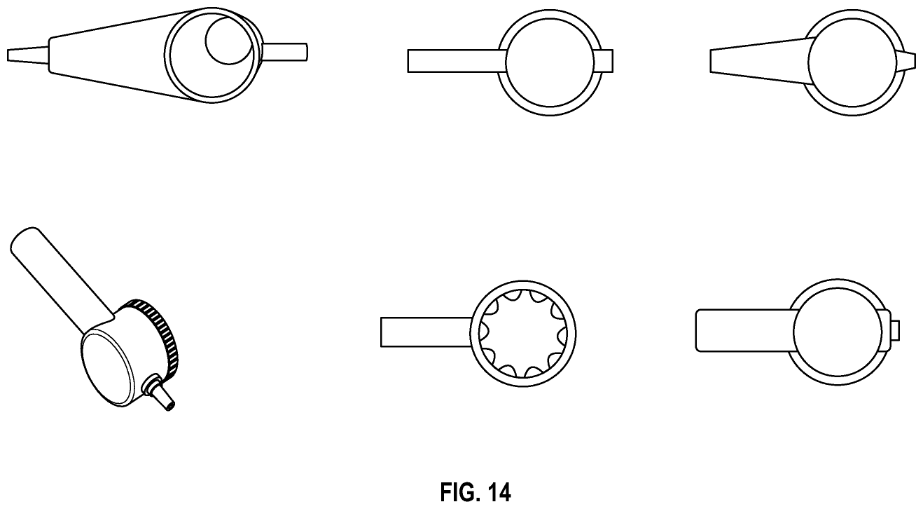

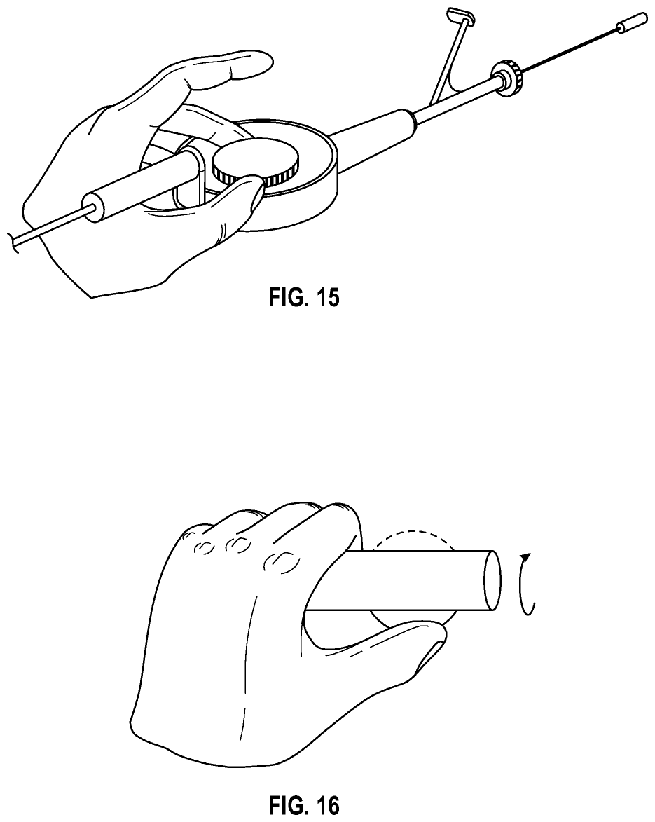

FIGS. 13-16 illustrate various handle and/or grips that can be used with the any of the guide extension advancement mechanisms and/or guide catheters described herein. Various grips can be used, including but not limited to, ball grip, bike handle grip, trigger handle grip, and/or a pencil grip. FIG. 13 illustrates examples of guide extension advancement mechanisms with a thumb wheel. The thumb wheel can allow for the operator to hold the handle of the guide extension advancement mechanism within their hand with a natural grip. FIG. 14 illustrates examples of guide extension advancement mechanisms with a finger wheel. The finger wheel can allow for the operator to grip the handle in their hand while actuating the wire and guide extension catheter by moving the wheel with their finger. FIG. 15 illustrates a handle grip that allows under body control while resting the housing on a hand grip. FIG. 16 illustrates a handle grip that can be rotated with a thumb or finger. For example, as illustrated in FIG. 16 the thumb of an operator can be used to push on a tab which will then rotate the handle 180 degrees to move any associated components, for example a wire and guide extension catheter.

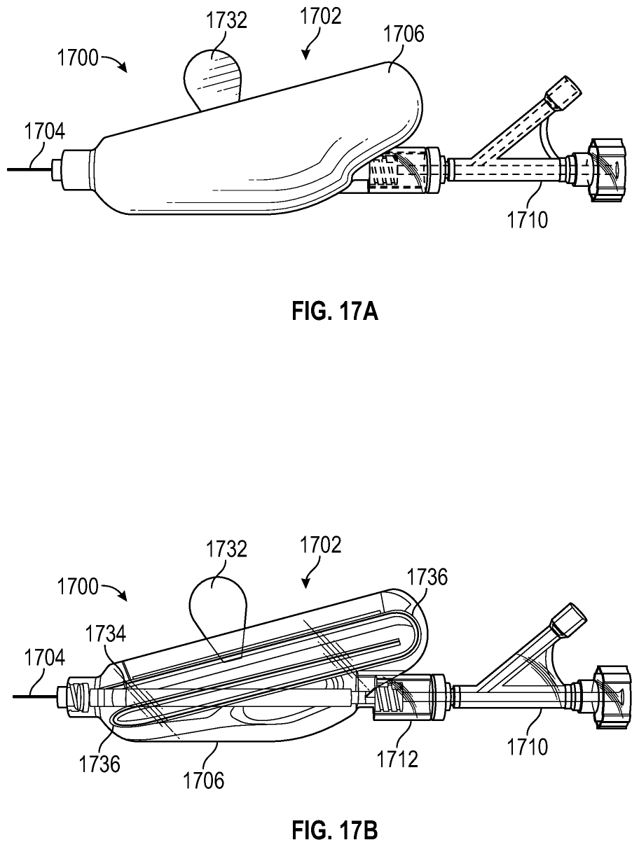

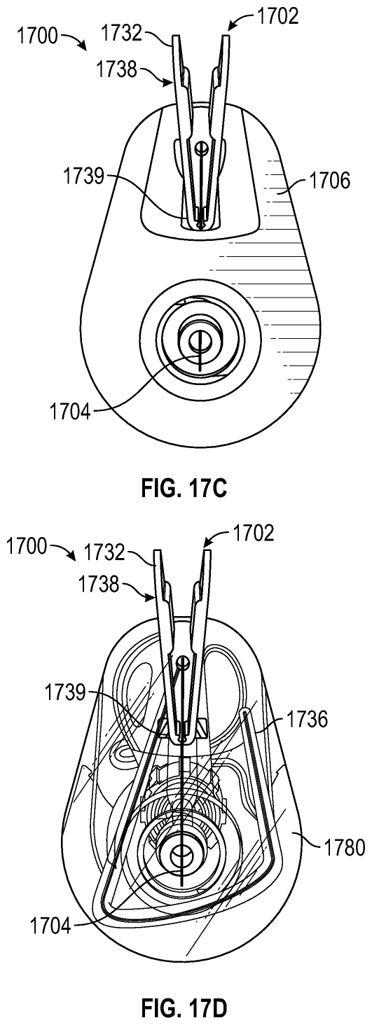

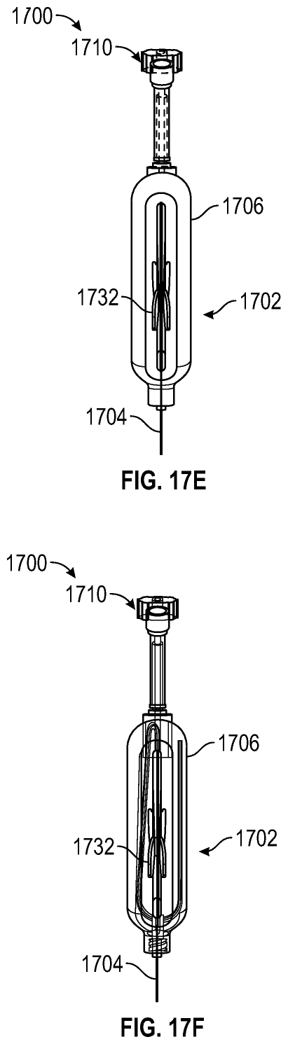

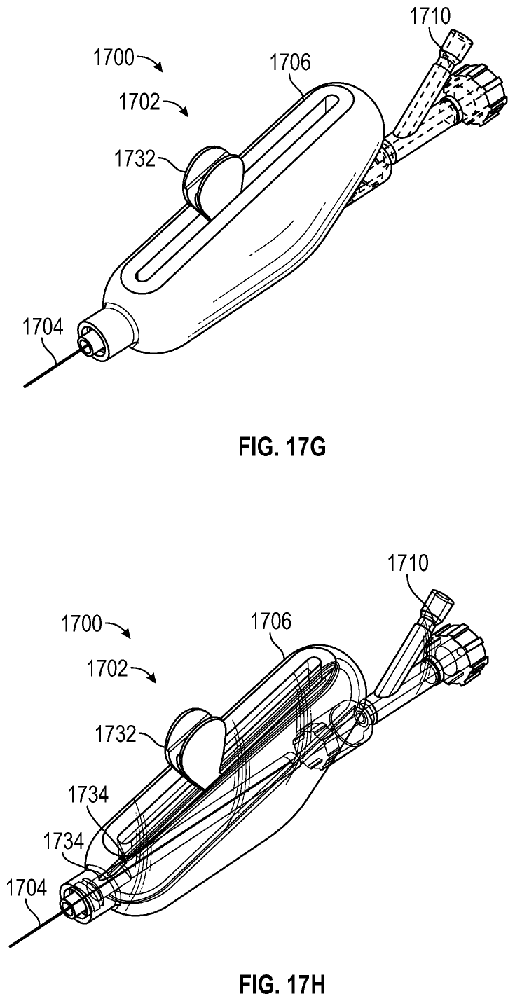

FIGS. 17A-22B illustrate examples of a catheter control system with a guide catheter, guide extension catheter, and a catheter control center that incorporates a guide extension advancement mechanism to actuate a guide extension catheter and wire within the guide catheter, a guide extension catheter wire, and a hemostatic valve. As described herein the guide extension catheter can be designed to be incorporated into the guide catheter as it is inserted into the patient and passed through the arteries.

FIGS. 17A-17H illustrate a catheter control system 1700 with a guide extension advancement mechanism 1702 to actuate a guide extension catheter (not shown) within the guide catheter (not shown). The guide extension advancement mechanism 1702 can include a housing 1706 with a finger pinch wire advancement 1732 that is used to advance or actuate a guide extension catheter wire 1704. FIGS. 17A-17B illustrate side views of the catheter control system 1700 and FIG. 17B shows the housing 1706 in transparent as to allow viewing of the inner components. The proximal end 1736 of guide extension catheter wire 1704 can be folded or bent within a wire channel 1780 within the housing as illustrated in FIG. 17B. In some cases, the guide extension catheter wire 1704 can have different properties or characteristics throughout the length of the wire. For example, the guide extension catheter wire 1704 can have different diameters at the proximal end that is folded or bent within the channel 1780 than the diameter of a more distal part of the guide extension catheter wire 1704 that is passed through the guide catheter. In some cases, the guide extension catheter wire 1704 can be more malleable or flexible at the proximal end that is folded or bent within the channel 1780 to allow the wire to move, fold, and bend within the channel. In contrast, the guide extension catheter wire 1704 can be less flexible at the more distal portions that are extended through the guide catheter to prevent the guide extension catheter wire 1704 from twisting, tangling, bending, or otherwise preventing movement of the guide extension catheter wire 1704 within the guide catheter. In some cases, the proximal end of the guide extension catheter wire 1704 can be a different shape than the guide extension catheter wire 1704 at the more distal end. In other cases, the guide extension catheter wire 1704 at the proximal end is the same shape and material as the guide extension catheter wire 1704 at the more distal ends of the device.

The guide extension advancement mechanism 1702 can also have a seal 1734 within the housing 1706 to prevent fluid or other contaminates from entering into the guide extension advancement mechanism 1702. In some cases, the seal 1734 can be a double seal or any seal necessary to prevent fluid ingress into the housing or any component of the mechanism. The catheter control system 1700 can include a hemostatic valve 1710 positioned proximal to the guide extension advancement mechanism 1702 and a swivel valve 1712 positioned between the hemostatic valve 1710 and the guide extension advancement mechanism 1702. The hemostatic valve 1710 can be used as described herein to deliver instruments or other wires through the guide catheter and/or guide extension catheter to the target area.

In some cases, the hemostatic valve 1710 and/or valve 1712 can be integrated into the guide extension advancement mechanism 1702 itself and can be formed as one piece. For example, the valve 1712 can be positioned on the distal end of the guide extension advancement mechanism 1702 and the hemostatic valve 1710 can be positioned on the proximal end of the guide extension advancement mechanism 1702. This arrangement can allow for the catheter control system 1700 to have the guide extension advancement mechanism 1702 with an integrated hemostatic valve 1710 and/or valve 1712 in one component to allow a length of the components that is similar to that of existing hemostatic valve systems.

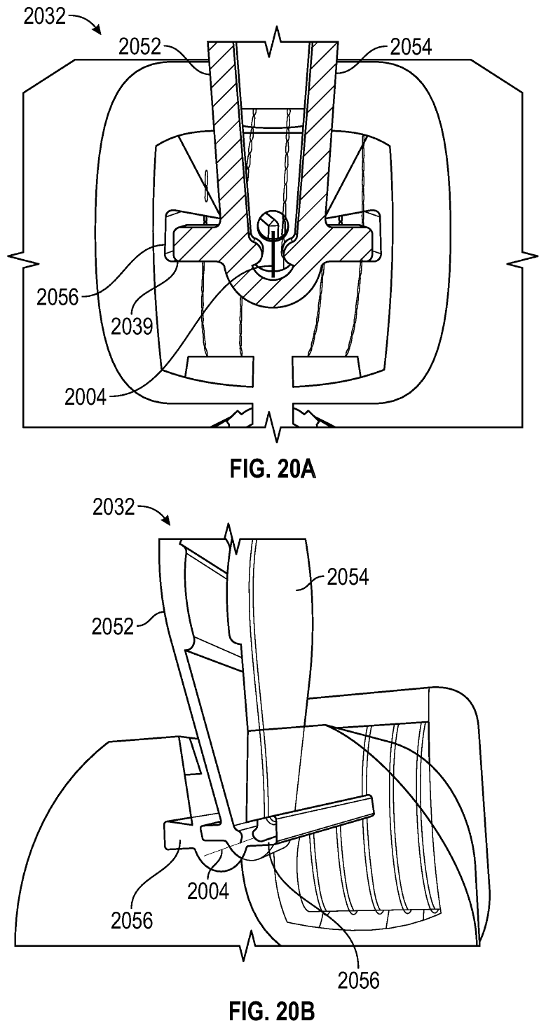

FIGS. 17C-17D illustrate front views of the catheter control system 1700 with a guide extension advancement mechanism 1702 with the guide extension catheter wire 1704 extending out of the device distally (out of the page). FIG. 17D shows the housing 1706 in transparent as to allow viewing of the inner components. As shown in FIGS. 17C-17D, the finger pinch advancement mechanism 1732 can have two tabs (described in more detail with reference to FIGS. 20A-20B) that can be pushed together at the top 1738 by the operator in order to pinch the guide extension catheter wire 1704 and move the guide extension catheter wire 1704. For example, when the tabs are pushed together at the top 1738, the bottom portions 1739 can pinch the guide extension catheter wire 1704 and move the wire in a proximal to distal or distal to proximal direction. When the tabs are not pushed together at the top 1738 (left separated), the top portions and bottom portions 1739 can be in a resting state with the tabs flexed outward and not exerting a force on the guide extension catheter wire 1704.

FIGS. 17E-17F illustrate top views of the catheter control system 1700 with a guide extension advancement mechanism 1702 with the guide extension catheter wire 1704 extending out of the device distally and the finger pinch wire advancement 1732 extending out of the page. FIG. 17F shows the housing 1706 in transparent as to allow viewing of the inner components.

FIGS. 17G-17H illustrate perspective views of the catheter control system 1700 with a guide extension advancement mechanism 1702. FIG. 17F shows the housing 1706 in transparent as to allow viewing of the inner components of the device. In some cases the guide extension catheter wire can be advanced for between about 5 cm and about 20 cm, about 10 cm and about 15 cm, or about 7 cm. In some cases, the storage capacity of the bend or folded wire within the housing can be between about 5 cm and about 40 cm, about 10 cm and about 35 cm, about 15 cm and about 30 cm, about 20 cm and about 25 cm, or about 20 cm of wire. In some cases, the guide extension advancement mechanism 1702 can comprise a length of between about 5 cm and about 30 cm, about 10 cm and about 25 cm, or about 15 cm and about 20 cm. In some cases, the guide extension advancement mechanism 1702 can comprise a width of between about 1 cm and about 20 cm, or about 5 cm and about 15 cm. In some cases, the guide extension advancement mechanism 1702 can comprise a height of between about 1 cm and about 20 cm, or about 5 cm and about 15 cm.

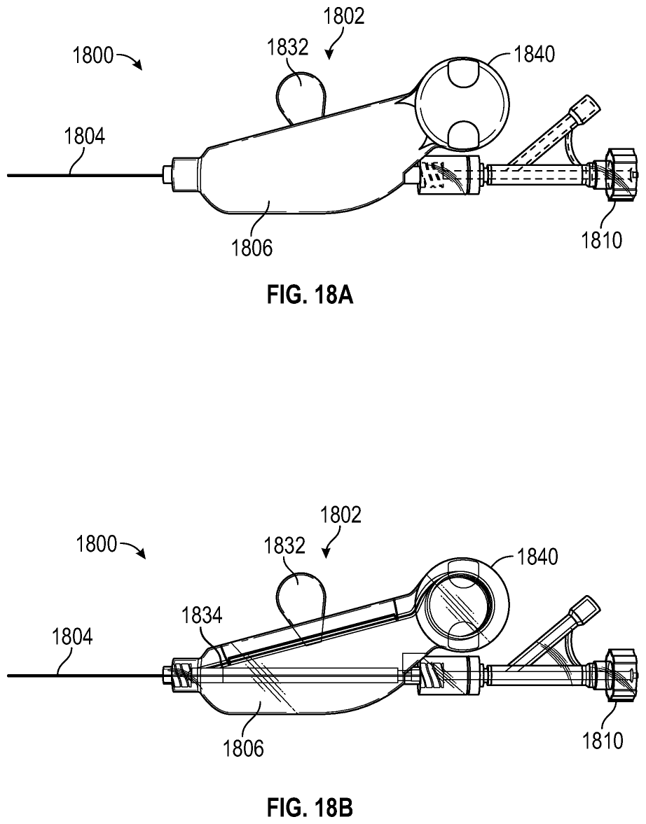

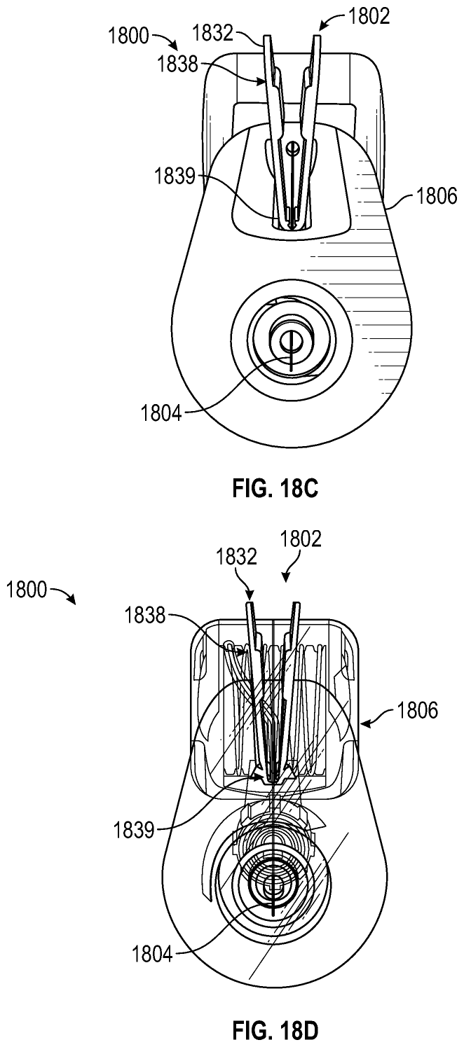

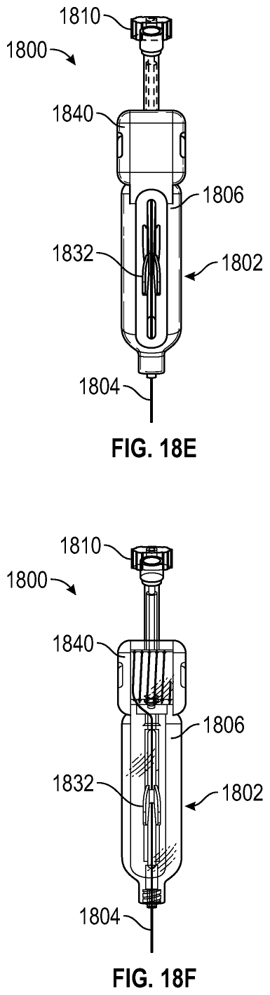

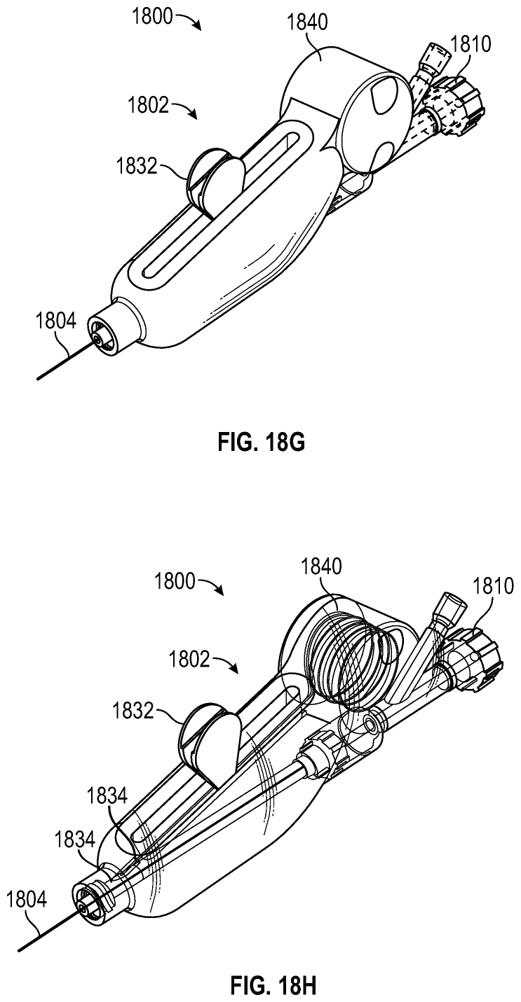

FIGS. 18A-18H illustrate a catheter control system 1800 with a guide extension advancement mechanism 1802 to actuate a guide extension catheter (not shown) within the guide catheter (not shown). The catheter control system 1800 of FIGS. 18A-18H is similar to the catheter control system 1700 of FIGS. 17A-17H. However, the catheter control system 1800 of FIGS. 18A-18H can use a spool mechanism 1840 to store the proximal end portion of the guide extension catheter wire 1804. The guide extension catheter wire 1804 is actuated to extend the guide extension catheter wire 1804 and the corresponding guide extension catheter (not shown) at the distal end of the guide extension catheter wire 1804. As the finger pinch advancement mechanism 1832 is pushed together at the top portions 1838, the guide extension catheter wire 1804 is pinched and the finger pinch advancement mechanism 1832 can be moved in a distal direction and the guide extension catheter wire is uncoiled from the spool mechanism 1840 to extend the guide extension catheter wire 1804 in the distal direction. In some cases, the spool can be used passively with finger advancement.

In some cases the guide extension catheter wire can be advanced for between about 5 cm and about 20 cm, about 10 cm and about 15 cm, or about 6 cm. In some cases, the spool mechanism can store between about 5 cm and about 60 cm, about 10 cm and about 55 cm, about 15 cm and about 50 cm, about 20 cm and about 45 cm, about 25 cm and about 40 cm, about 30 cm and about 35 cm, or about 36 cm of wire. In some cases, the guide extension advancement mechanism 1802 can comprise a length of between about 1 cm and about 30 cm, about 5 cm and about 25 cm, or about 10 cm and about 20 cm. In some cases, the guide extension advancement mechanism 1802 can comprise a width of between about 1 cm and about 20 cm, or about 5 cm and about 15 cm. In some cases, the guide extension advancement mechanism 1802 can comprise a height of between about 1 cm and about 20 cm, or about 5 cm and about 15 cm.

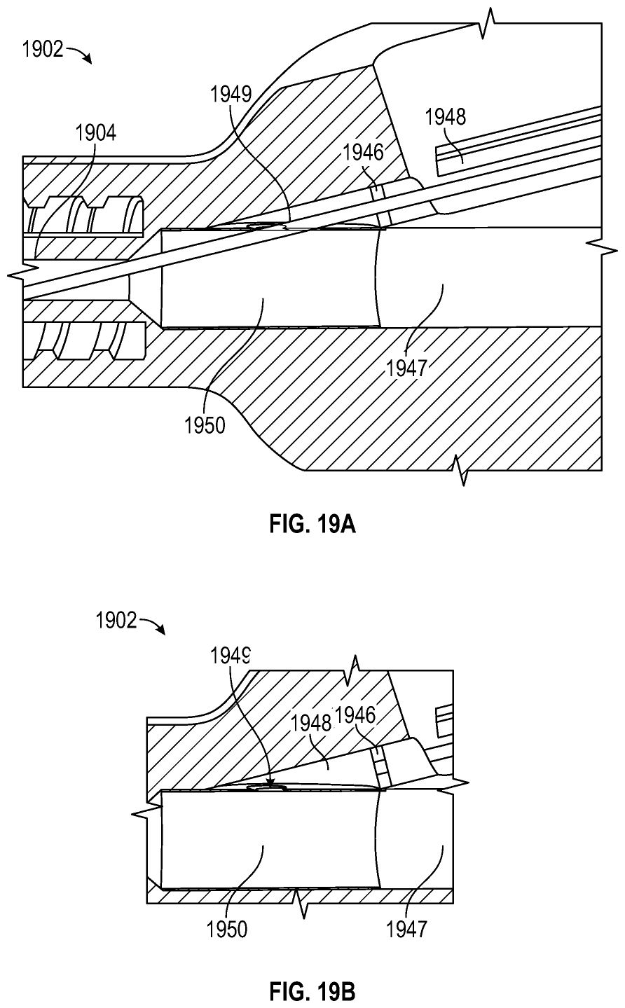

FIGS. 19A-19B illustrate an embodiment of an interior of a guide extension advancement mechanism 1902. As illustrated in FIGS. 19A-19B the guide extension advancement mechanism 1902 can include a double seal system. The guide extension advancement mechanism 1902 can have a first seal 1946 positioned on the outer surface of a main channel 1947 between the main channel 1947 and a guide extension catheter wire channel 1948 to seal the opening that the guide extension catheter wire 1904 passes through. The first seal 1946 can prevent fluid or other contaminates from entering the guide extension catheter wire channel 1948. The guide extension advancement mechanism 1902 can have a second seal 1949 positioned within the guide extension catheter wire channel 1948. The second seal 1949 can be perpendicular to the guide extension catheter wire 1904. The second seal 1949 can be a dynamic radial seal that seals against the guide extension catheter wire 1904. The main channel 1947 can include a passive filter 1950 that can be used to prevent pooling of fluid or other contaminates in the branch between the main channel 1947 and the guide extension catheter wire channel 1948.

FIGS. 20A-20B illustrate a zoomed in view of a finger pinch wire advancement 2032. The finger pinch wire advancement 2032 can be a flexible, plastic finger-pinching grip. As shown in FIG. 20A, the resting state of the finger pinch wire advancement 2032 is for the tabs 2052, 2054 to be flexed outward (as shown by the arrows in FIG. 20A). As the tabs 2052, 2054 are pushed inward as shown in FIG. 20B, the wire 2004 can be pinched by the bottom portion 2039 of the finger pinch wire advancement 2032. The finger pinch wire advancement 2032 can then be moved in a distal direction along a track 2056 which will also move the wire 2004 that is pinched by the finger pinch wire advancement 2032. The finger pinch wire advancement 2032 can then be released and moved back to a resting state. The wire advancement can be repeated as needed to move the guide extension catheter wire in a distal to proximal or a proximal to distal direction within the catheter control system.

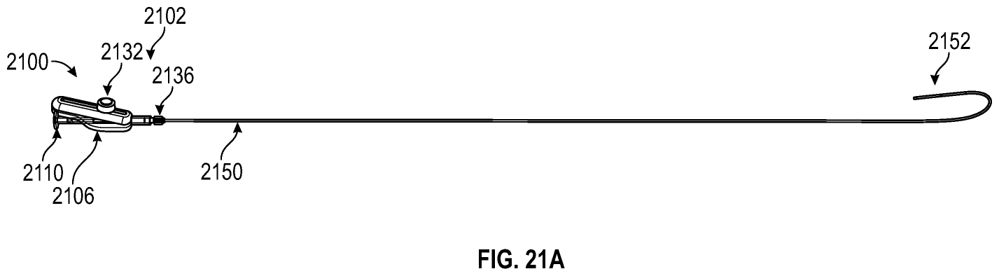

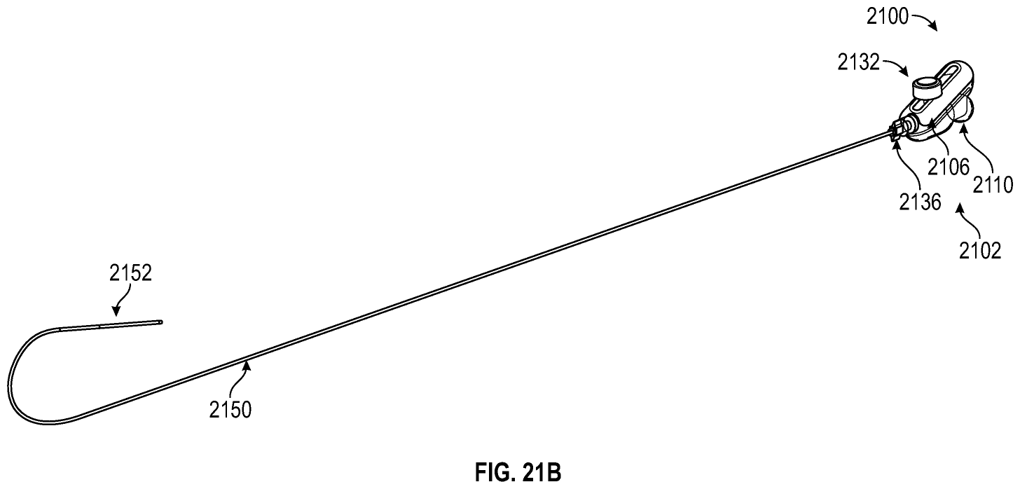

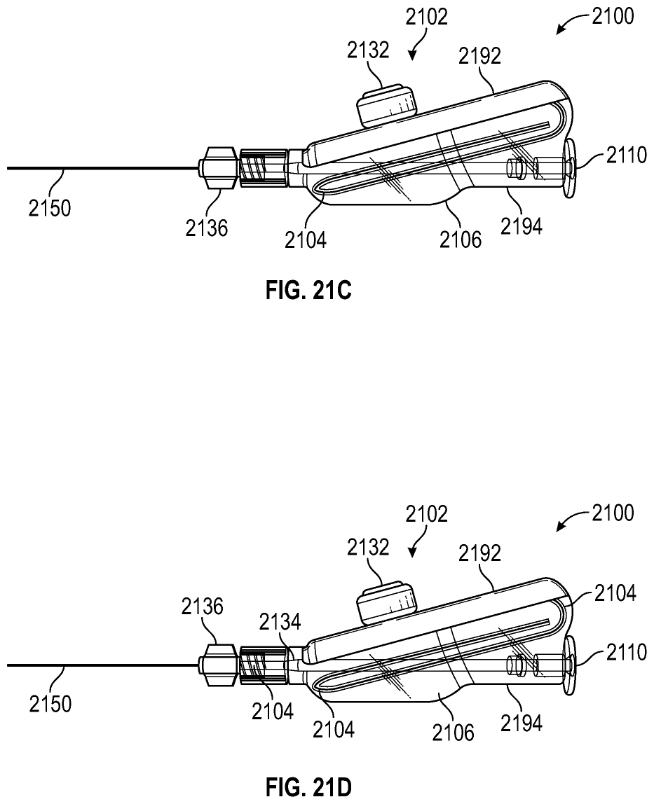

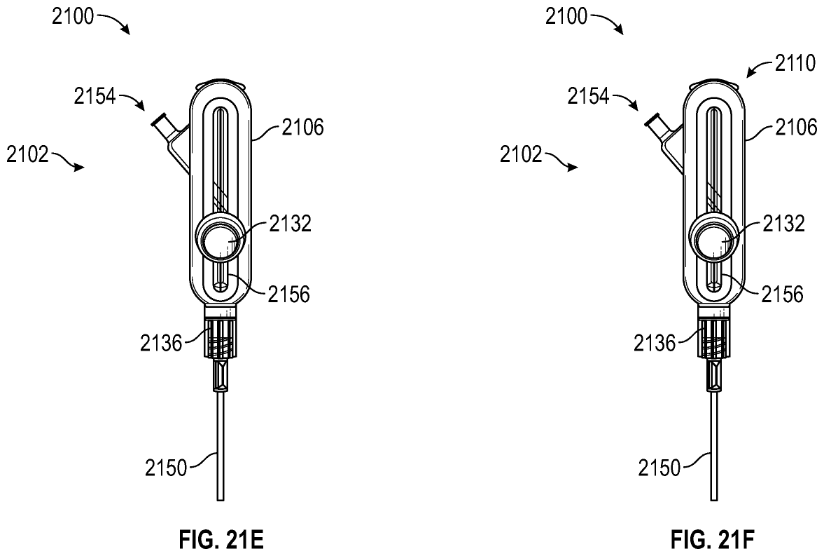

FIGS. 21A-21F illustrate a catheter control system 2100 with a guide extension advancement mechanism 2102 to actuate a guide extension catheter (not shown) within the guide catheter (not shown). The catheter control system 2100 of FIGS. 21A-21F is similar to the catheter control system 2100 of FIGS. 17A-17H and 18A-18H. However, the catheter control system 2100 of FIGS. 21A-21F can use a finger knob advancement mechanism 2132 to actuate the guide extension catheter wire 2104 (shown in FIGS. 21C and 21D).

The guide extension catheter wire 2104 is actuated to extend the guide extension catheter wire 2104 and the corresponding guide extension catheter 2152 at the distal end of the guide extension catheter wire 2104 from the distal end of the guide catheter 2150. Additionally, the catheter control system 2100 of FIGS. 21A-21F also incorporates the functionality of the hemostatic valve 2110. Further, the catheter control system 2100 of FIGS. 21A-21F incorporates a rotating connector 2136 within the housing 2106 of the catheter control center of the finger knob advancement mechanism 2132. The rotating connector 2136 can allow the guide catheter 2150 to rotate independent of the remainder of the catheter control system 2100. This feature can allow the guide catheter to be able to rotate independent of the components of the catheter control center and enable movement of the guide catheter during and post insertion This feature also allows a practitioner to rotate the catheter control center without moving the guide catheter, resulting in greater flexibility of equipment positioning throughout a high-intensity, time-critical procedure.

The integration of the hemostatic valve 2110 and the valve 2136 within the housing 2106 of the catheter control center can allow for an integrated and easy to use device that the operator can control during use.

FIGS. 21A-21B illustrate views of the catheter control system 2100 with a finger knob advancement mechanism 2132. FIGS. 21C-21D illustrate a side view of the catheter control system 2100 with a finger knob advancement mechanism 2132. The housing 2106 is shown in transparent to allow visibility of the interior components within the housing 2106. The housing 2106 can have two compartments, the guide extension catheter wire compartment 2192 and the main valve compartment 2194. The guide extension catheter wire 2104 can be seen in a double bend within the guide extension catheter wire compartment 2192 of the housing 2106 as described with reference to FIGS. 17A-17H. The guide extension catheter wire compartment 2192 can accommodate the guide extension catheter wire 2104 and the advancement mechanism 2132. In some cases, the main valve compartment 2194 can act similar to a hemostatic valve described herein and can allow for all other wires and/or devices to pass through the main valve compartment 2194 from the valve to the guide catheter. In some embodiments, the guide extension catheter wire compartment 2192 and the main valve compartment 2194 can be connected by a seal 2134. The seal 2134 can prevent fluid from entering the guide extension catheter wire compartment 2192.

FIG. 21E-21F illustrates a top view of the catheter control system 2100 with a finger knob advancement mechanism 2132. The finger knob advancement mechanism 2132 can move along a track 2156 as illustrated in FIGS. 21E-21F to actuate the guide extension catheter wire 2104 (shown in FIGS. 21C and 21D) and the guide extension catheter 2152 (shown in FIGS. 21A-21B). FIGS. 21E-21F illustrates the side port 2154 which can be incorporated into the housing 2106. The side port 2154 can be used to provide additional support. For example, the side port 2154 can be used to flush the catheter, attach a manifold, and/or measure pressure or take other measurements. Although, the side port 2154 is shown on the side of the housing 2106, the port 2154 can be positioned on any portion of the housing 2106. Additionally, the hemostatic valve 2110 is shown on the proximal end of the housing 2106. However, the hemostatic valve 2110 can be positioned on any portion of the housing 2106 that allows the instrumentation or other devices to be delivered through the valve and/or into the guide catheter 2150.

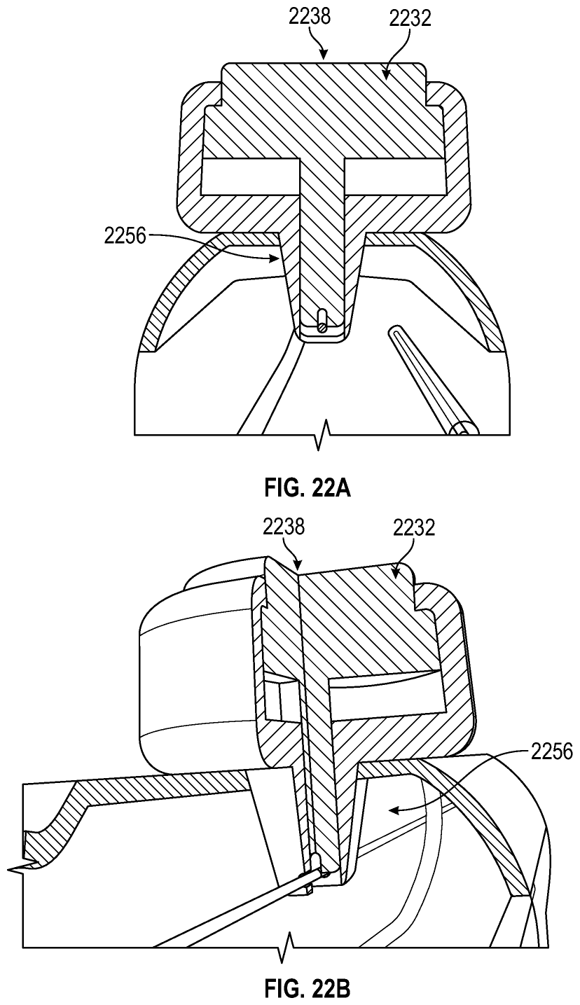

FIGS. 22A-22B illustrates the finger knob advancement mechanism 2232 that can be used to actuate the guide extension catheter wire 2104 (shown in FIGS. 21C and 21D) and move the guide extension catheter 2152. As the finger knob advancement mechanism 2232 is depressed at the top portions 2238 to move the finger knob advancement mechanism 2232 along the track 2156. For example, the finger knob advancement mechanism 2232 can be depressed and moved along the track 2156 in a distal direction and the guide extension catheter wire can be moved within the housing to extend the guide extension catheter wire in the distal direction. The finger knob advancement mechanism 2232 can have a round knob slider with a spring-loaded push-button as shown in FIGS. 22A-22B. In the default state (released or not depressed state) the finger knob advancement mechanism 2232 is un-pinched from the guide extension catheter wire. Once the top portion 2238 is depressed, the finger knob advancement mechanism 2232 can pinch and grab onto the guide extension catheter wire for advancement or retraction of the guide extension catheter wire. In some cases, the outer shape of the catheter control center can be designed to be placed or rested on a table and to be easy to grab or manipulate by the operator. The catheter control center can be gripped easily with one hand so that the second hand can be used to hold another device or is otherwise free. In some cases, the catheter control system can include finger indentations to indicate how the operator is to hold the device. In some cases, the catheter control center can be weighted on the main valve compartment side to promote a certain orientation. In some cases, the catheter control system can have legs or a tacky or sticky underside of the catheter control center to allow the catheter control center to stay in one spot.

### Different Configurations of Guide Catheter Shafts

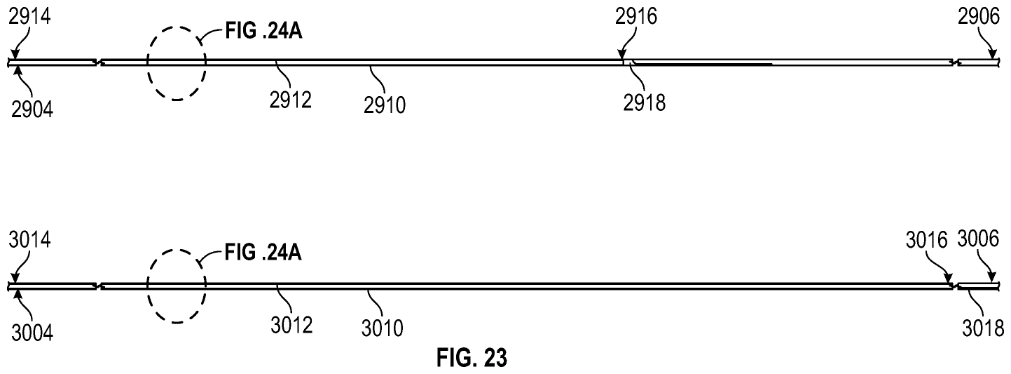

FIG. 23 illustrates cross-sections of three example configurations of a guide catheter shaft 2910, 3010 with a telescoping guide extension catheter 2912, 3012. The guide catheter shafts 2910, 3010 and the telescoping guide extension catheters 2912, 3012 can be the same as or similar to any of the guide catheter shafts and the telescoping guide extension catheters as described herein. The guide catheter shafts 2910, 3010 can be used in any vascular artery.

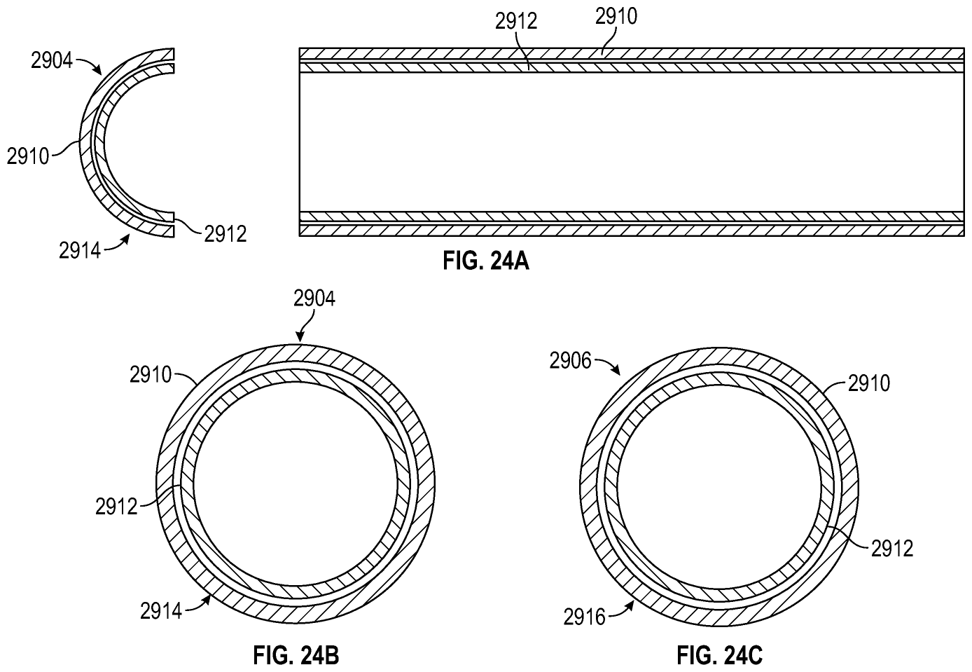

FIGS. 24A-24C illustrate an example implementation of a guide catheter shaft 2910 integrated with a telescoping guide extension catheter 2912. In this implementation, the guide catheter 2910 can have a constant wall thickness extending from a distal end 2904 to a proximal end 2906. For example, the wall thickness can be between about 0.1 mm and about 3 mm, about 0.5 mm and about 2.5 mm, about 1.0 mm and about 2.0 mm, or about 0.125 mm. The guide extension catheter 2912 can have a constant wall thicknesses extending from a distal end 2914 of a shaft of the guide extension catheter 2912 to a proximal end 2916 of the shaft of the guide extension catheter 2912. For example, the wall thickness can be between about 0.05 mm and about 3 mm, about 0.5 mm and about 2.5 mm, about 1.0 mm and about 2.0 mm, or about 0.1 mm. In the illustrated configuration, the wall thickness of the guide catheter 2910 can be greater than the wall thickness of the guide extension catheter 2912. In other configurations, the wall thickness of the guide catheter 2910 can be less than the wall thickness of the guide extension catheter 2912.

In some configurations, the guide extension catheter 2912 can have a diameter that is less than an inner diameter of the guide catheter 2910 such that the guide extension catheter 2912 can be positioned within the guide catheter 2910. For example, an inner diameter of the guide extension catheter 2912 can be between about 0.5 mm and about 5 mm, about 1.0 mm and about 4.5 mm, about 1.5 mm and about 4.0 mm, about 2.0 mm and about 3.5 mm, about 2.5 mm and about 3.0 mm, or about 1.6 mm.

In some aspects, the guide extension catheter 2912 can include a transition region 2918. At the transition region 2918, a wire of the guide extension catheter 2912 can transition into the shaft of the guide extension catheter 2912.

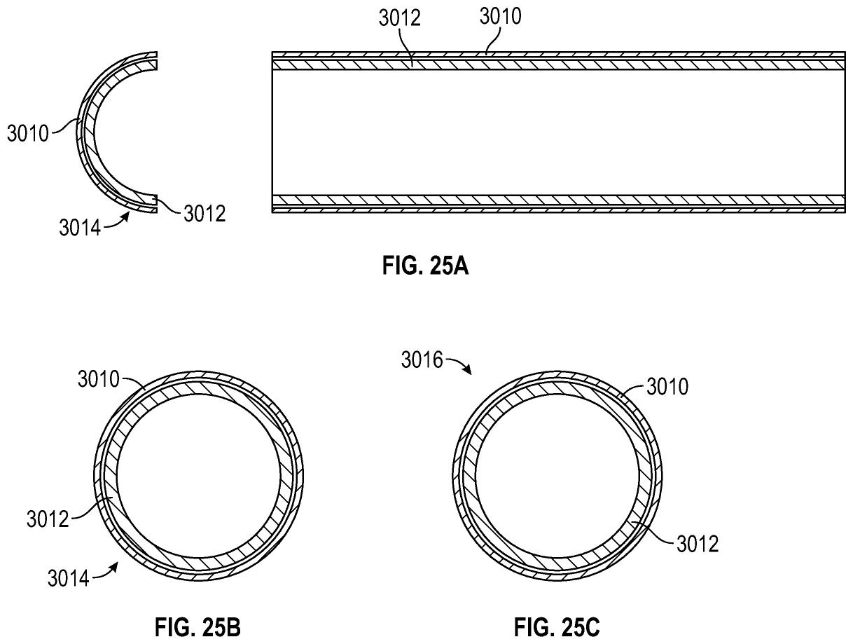

FIGS. 25A-25C illustrate an example implementation of a guide catheter shaft 3010 integrated with a telescoping guide extension catheter 3012. In this implementation, the guide catheter 3010 can have a constant wall thickness extending from a distal end 3004 to a proximal end 3006. For example, the wall thickness can be between about 0.01 mm and about 3 mm, about 0.5 mm and about 2.5 mm, about 1.0 mm and about 2.0 mm, or about 0.065 mm. The guide extension catheter 3012 can have a constant wall thicknesses extending from a distal end 3014 of a shaft of the guide extension catheter 3012 to a proximal end 3016 of the shaft of the guide extension catheter 3012. For example, the wall thickness can be between about 0.05 mm and about 3 mm, about 0.5 mm and about 2.5 mm, about 1.0 mm and about 2.0 mm, or about 0.125 mm. In some configurations, the wall thickness of the guide catheter 3010 can be greater than the wall thickness of the guide extension catheter 3012. In the illustrated configuration, the wall thickness of the guide catheter 3010 can be less than the wall thickness of the guide extension catheter 3012.

In some configurations, the guide extension catheter 3012 can have a diameter that is less than an inner diameter of the guide catheter 3010 such that the guide extension catheter 3012 can be positioned within the guide catheter 3010. For example, an inner diameter of the guide extension catheter 3012 can be between about 0.5 mm and about 5 mm, about 1.0 mm and about 4.5 mm, about 1.5 mm and about 4.0 mm, about 2.0 mm and about 3.5 mm, about 2.5 mm and about 3.0 mm, or about 1.67 mm.

As shown in FIG. 23, the guide extension catheter 3012 can include a transition region 3018. At the transition region 3018, a wire of the guide extension catheter 3012 can transition into the shaft of the guide extension catheter 3012.

### Configurations of Failsafe Mechanisms

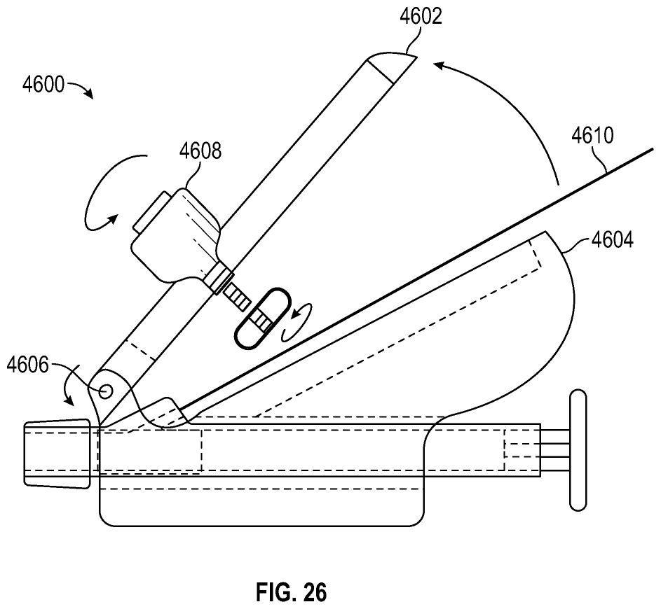

FIGS. 26-28 illustrate configurations of failsafe mechanisms configured to attach to a valve (e.g., a hemostatic valve) and allow the user to manually manipulate the guide extension catheter wire in the event the actuation mechanism fails. The failsafe mechanism can be used with an actuation mechanism and catheter control center described herein. For example, the failsafe mechanisms can be configured to release the actuation mechanism if the user needs to manually manipulate the wire of the guide extension catheter. Moreover, the failsafe mechanisms can be configured to allow a user to remove a guide extension catheter from the guide catheter if the guide extension catheter is stuck, if more room is needed within the guide catheter, or for other reasons. FIG. 26 illustrates a configuration of a failsafe mechanism 4600 used with a clamshell type actuation mechanism. The actuation mechanism 4600 can include a first portion 4602 and a second portion 4604. The first portion 4602 can be coupled to the second portion 4604 via a hinge 4606 at a first end of the actuation mechanism 4600. The first portion 4602 and the second portion 4604 can be removably coupled via coupling mechanism 4608. If the user needs to manually manipulate the wire 4610, the user can disengage the coupling mechanism 4608 such that the first and second portions 4602, 4604 can be separated. As illustrated in FIG. 26, the coupling mechanism 4608 can be a button that can be twisted or unscrewed to uncouple the first portion 4602 and the second portion 4604. In some configurations, the coupling mechanism 4608 can be configured to move the guide extension catheter in a proximal direction and a distal direction within the guide catheter.

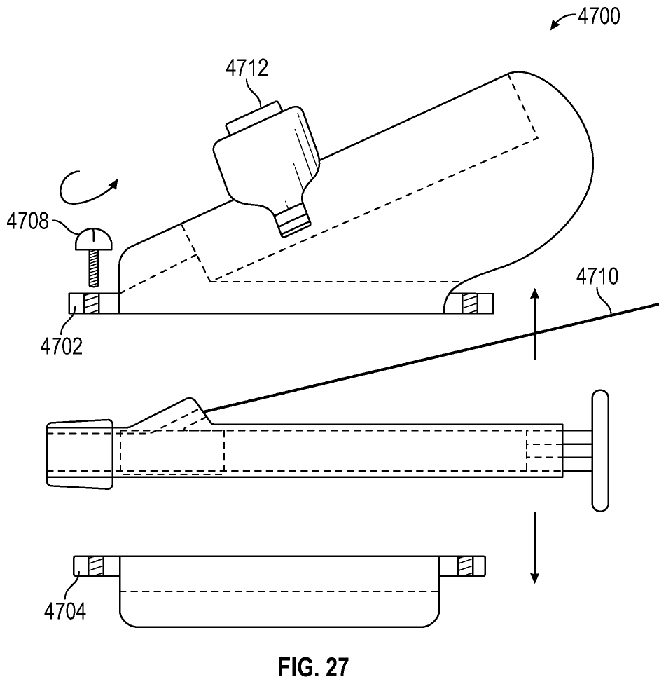

FIG. 27 illustrates a configuration of a failsafe mechanism 4700. The failsafe mechanism 4700 can include a first portion 4702 and a second portion 4704 configured to be separable from the first portion 4702. The first portion 4702 and the second portion 4704 can be removably coupled via coupling mechanism 4708 (e.g., a screw). If the user needs to manually manipulate the wire 4710, the user can disengage the coupling mechanism 4708 (e.g., unscrewing a screw) such that the first and second portions 4702, 4704 can be separated. The failsafe mechanism 4700 can include an actuation mechanism 4712 configured to move the guide extension catheter in a proximal direction and a distal direction within the guide catheter.

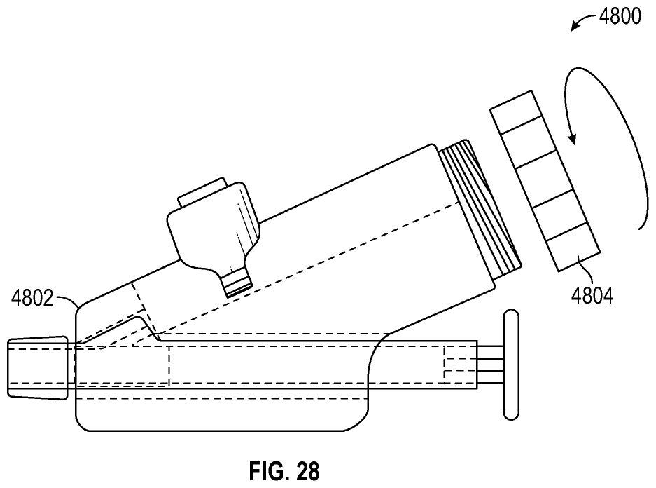

FIG. 28 illustrates a configuration of a failsafe mechanism 4800. The failsafe mechanism 4800 can include an actuation mechanism with a first portion 4802 and a second portion 4804 configured to be separable from the first portion 4802. For example, the second portion 4804 can comprise a cap 4804 and the first portion 4802 can included a threaded portion configured to engage with the cap 4804. If the user needs to manually manipulate the wire (not shown), the user can disengage and remove the cap of the second portion 4804 from the first portion 4802.

### Different Configurations of Wire Storage Mechanisms

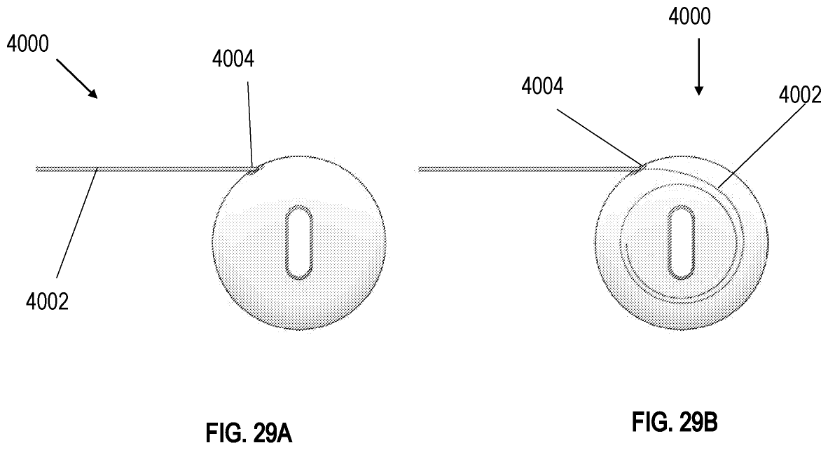

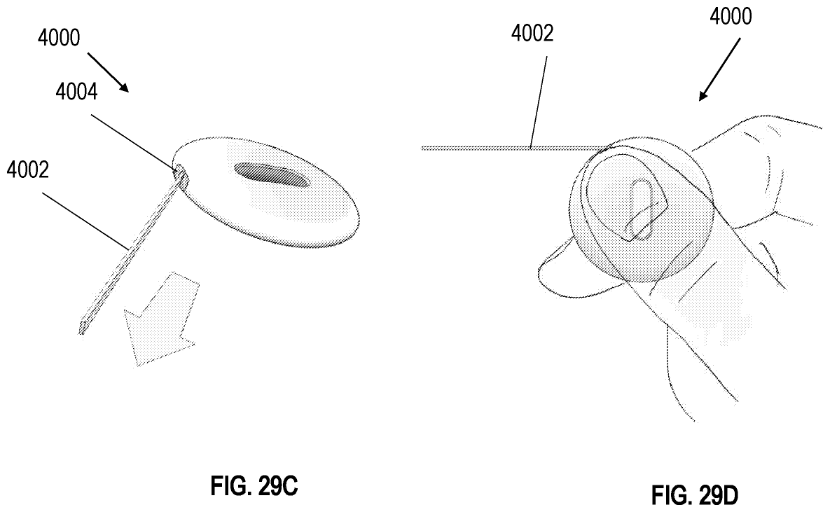

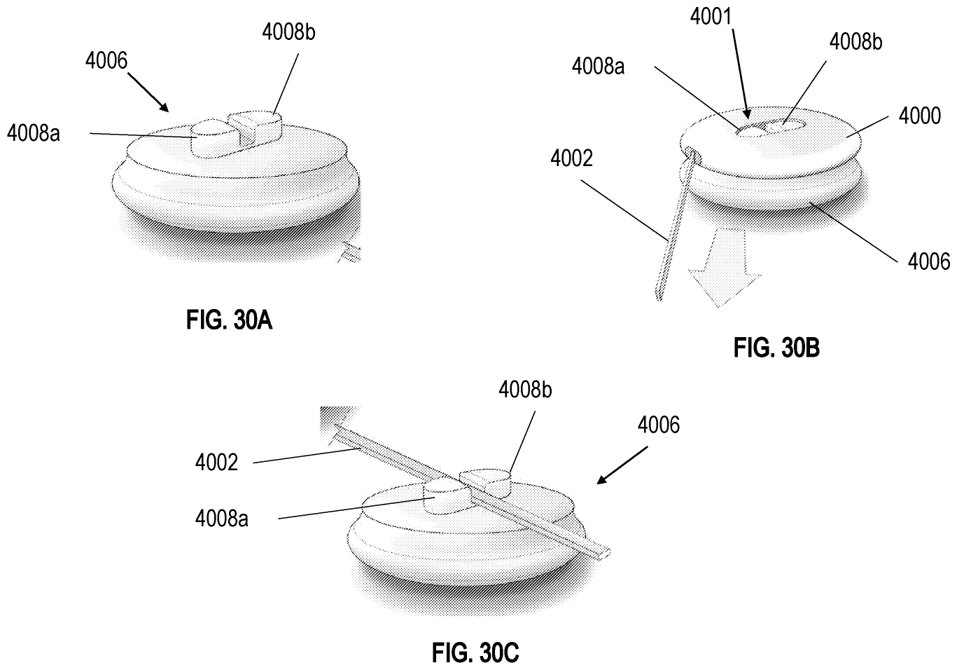

In some embodiments, the catheter control system can be replaced with an additional wire port on the hemostatic valve for the guide extension catheter wire. The wire port allows for the guide extension catheter and/or the guide catheter wire to remain separate to reduce wire confusion and entanglement. The wire port could be built distal or proximal to the hemostatic valve seal. If the wire port is distal to the valve seal, the same scaling technology (or equivalent) discussed above could be applied. If the wire port is proximal to the valve seal, no additional scaling would be required. The guide extension catheter wire port can be configured to accept the guide extension catheter wire and maximize efficiency of wire movement. In some embodiments, the catheter control system can consist of the additional wire port on the hemostatic valve described above as well as a wire storage mechanism. FIGS. 29A-36B illustrate different configurations of wire storage mechanisms and assorted accessories. This embodiment can prevent extra wire from cluttering the limited space for the practitioner. As further discussed below in relation to FIGS. 30A-30C, an anchor 4006 can offer the practitioner the option to anchor the guide extension catheter wire to the table, thereby preventing unintended movement of the guide extension wire.

FIGS. 29A-29D illustrate a compact spool mechanism 4000 configured to store a wire 4002. The compact spool mechanism 4000 can have a bend radius of between about 2 mm and about 20 mm, about 5 mm and about 15 mm, or about 10 mm. The compact spool mechanism 4000 can have a total diameter of between about 0.5 inches and about 5 inches, about 1 inch and about 4 inches, about 2 inches and about 3 inches. The compact spool mechanism 4000 can have a friction opening 4004 configured to increase the resistance on the wire 4002 when the wire 4002 is pulled through the opening 4004 or retracts through opening 4004. In use, wire 4002 can be stored within the spool mechanism 4000 (FIG. 29B). When a user needs more length, the user can pull the wire 4002 from the spool mechanism 4000 (FIG. 29C). When a pulling force is not being applied to the wire 4002, the wire 4002 can retract into the spool mechanism 4000.

FIGS. 30A-30C illustrate an example anchor 4006. The anchor 4006 can include a friction pad on a bottom surface of the anchor 4006. The anchor 4006 can be configured to weigh down the spool mechanism 4000 on a surface in use. Advantageously, the anchor 4006 can allow a user to pull the wire 4002 from the spool mechanism 4000 without needing to hold or otherwise handle the spool mechanism 4000. In some configurations, the anchor 4006 can include one or more prongs 4008a. 4008b. The illustrated configuration of the anchor 4006 has two prongs 4008a, 4008b. As shown in FIG. 30B, the spool mechanism 4000 can be attached to the anchor 4006. For example, the spool mechanism 4000 can include an opening 4001. In some aspects, the opening 4001 can be configured to receive the one or more prongs 4008a, 4008b. Alternatively, as shown in FIG. 30C, the wire 4002 can be pulled through a portion of the anchor 4006. In some aspects, the prongs 4008a, 4008b are configured to be moveable. For example, the prongs 4008a, 4008b can be moved toward one another to tighten a grip on the wire 4002. Alternatively, the prongs 4008a. 4008b can be moved apart to loosen a grip on the wire 4002.

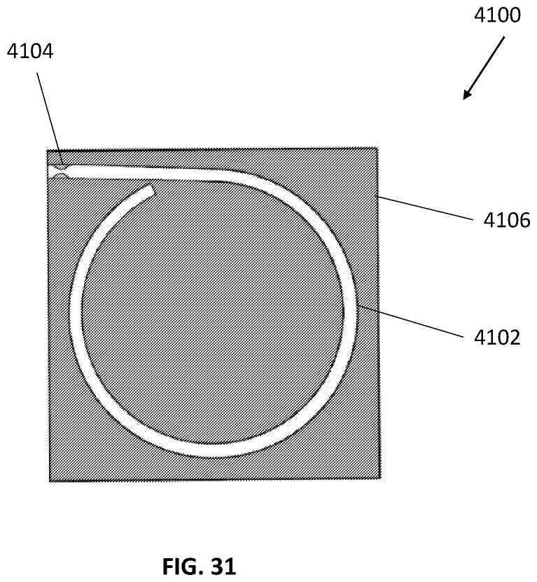

FIG. 31 illustrates a configuration of a spool mechanism 4100. Similar to the spool mechanism 4000, the spool mechanism 4100 can be configured to store a wire 4102. The spool mechanism 4100 can include a housing 4106. The housing 4106 can have a length of between about 5 cm and about 20 cm, about 10 cm and about 15 cm, or about 8 cm. The housing 4106 can have a width of between about 5 cm and about 20 cm, about 10 cm and about 15 cm, or about 8 cm. The housing 4106 can include a friction opening 4104 configured to keep to keep the wire 4102 in place when the user is not pulling the wire 4102 or when the wire 4102 is not retracting into the spool mechanism 4100. The wire 4102 can form a single loop within the housing 4106. For example, the loop can have a diameter of between about 1 cm and about 20 cm, about 5 cm and about 15 cm, or about 4 cm. In some configurations, the wire 4102 can form multiple loops within the housing 4106.

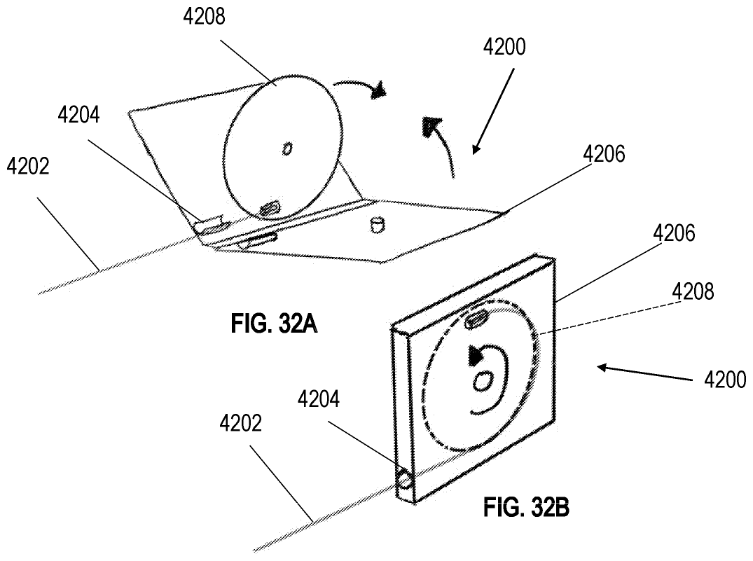

FIGS. 32A-32B illustrate a configuration of a spool mechanism 4200. The spool mechanism 4200 can include a housing 4206 configured to be opened (FIG. 32A) and closed (FIG. 32B). The spool mechanism 4200 can include a spool 4208 stored within the housing 4206. The spool 4208 can be configured to store a wire 4202. In use, the wire 4202 can be pulled through an opening 4204 of the housing 4206. When a user pulls the wire 4202 from the housing 4206, the spool 4208 can rotate a first direction. When the wire 4202 is being retracted into the housing 4206, the spool 4208 can rotate the opposite direction. In some configurations, the spool 4208 can be disposable. Advantageously, the housing 4206 can be opened and closed for case of loading a new spool 4208 and removing an old spool 4208. In some configurations, the spool 4208 can be reusable.

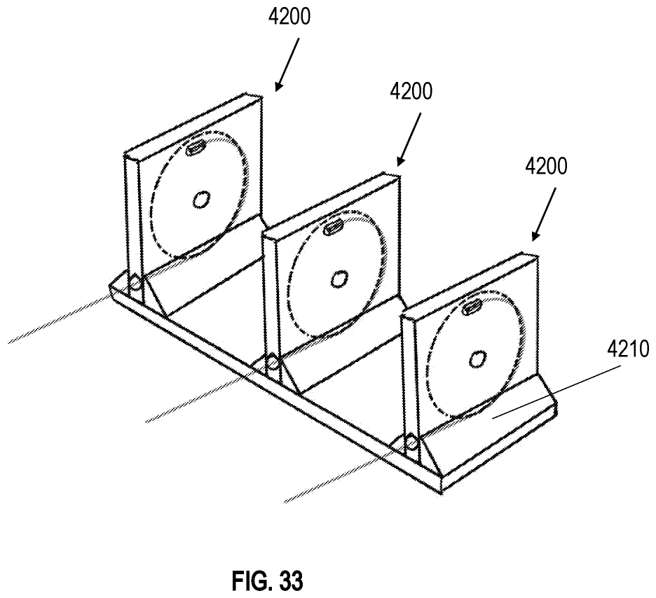

FIG. 33 illustrates a spool base 4210. The spool base 4210 can be used to combine multiple spool mechanisms 4200. For example, the illustrated configuration shows three spool mechanisms 4200. In some configurations, the spool base 4210 can hold two spool mechanisms 4200 or more than three spool mechanisms 4200 (e.g., four, five, six, seven).

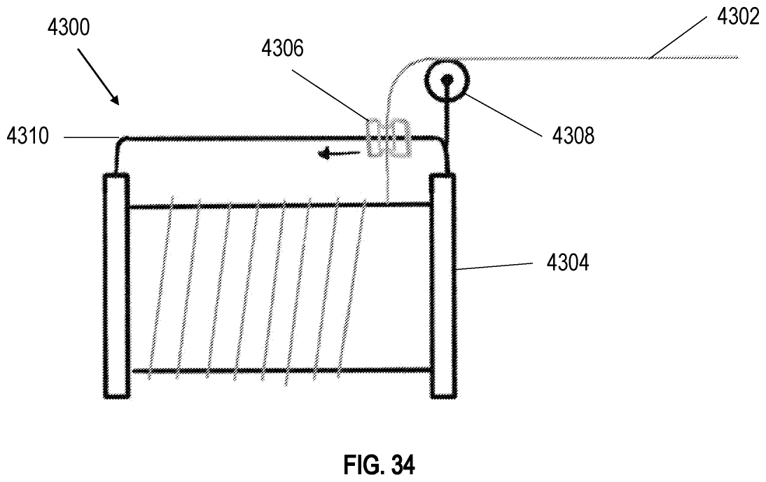

FIG. 34 illustrates a configuration of a spool mechanism 4300. The spool mechanism 4300 can include a spool 4304, a sliding guide 4306, a sliding rail 4310, and a pulley 4308. A wire 4302 can be wrapped around the spool 4304, pulled through the sliding guide 4306, and over the pulley 4308. The sliding guide 4306 can be positioned on the sliding rail 4310 such that the sliding guide 4306 can be moved along the length of the sliding rail 4310.

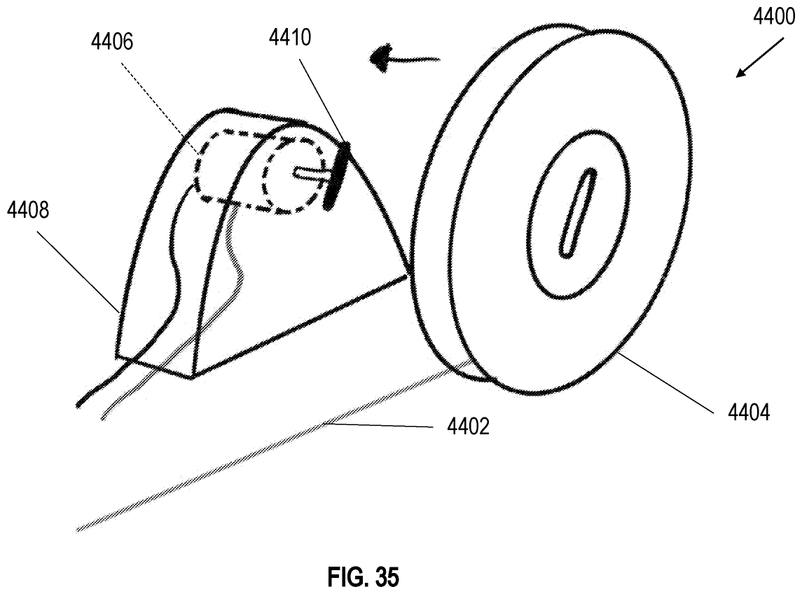

FIG. 35 illustrates a configuration of a spool mechanism 4400. The spool mechanism 4400 can include a spool 4404 and a base 4408. The spool 4404 can be configured to store a wire 4402. The base 4408 can include a motor 4406 with an attachment portion 4410. The spool 4404 can be configured to attach to the attachment portion 4410 of the motor 4406. The motor 4406 can be configured to provide retraction and adjustable drag when the wire 4402 is being pulled from the spool 4404. In some configurations, the base 4408 can be configured to be reusable. In some configurations, the spool 4404 can be configured to be disposable.

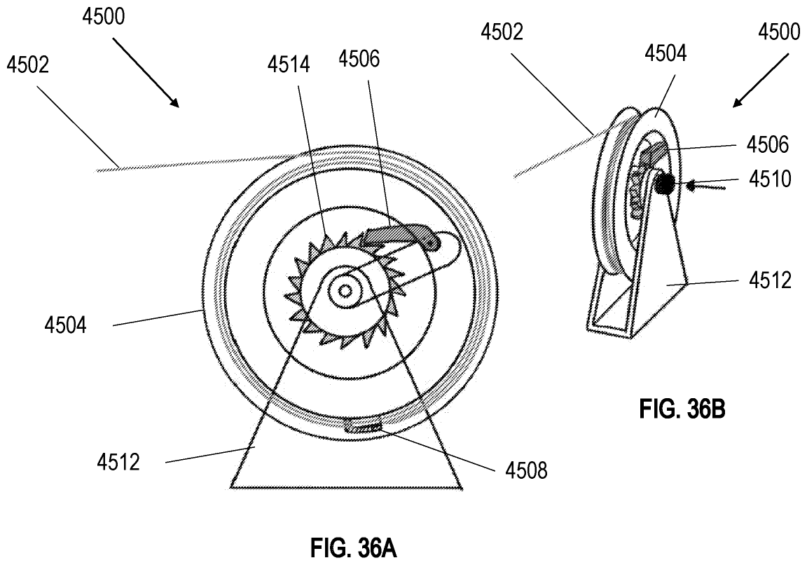

FIGS. 36A and 36B illustrate a configuration of a spool mechanism 4500. The spool mechanism 4500 can include a spool 4504, a base 4512, a ratchet 4506, and a ratchet wheel 4514. One end of a wire 4502 can be received by a wire stop 4508 of the spool 4504 and wrapped around the spool 4504. The ratchet 4506 and the ratchet wheel 4514 can be positioned radially inward of the wire 4502. The spool mechanism 4500 can be configured to allow the wire 4502 to be pulled in one direction. The base 4512 can include a release button 4510 configured to disengage the ratchet 4506 from the ratchet wheel 4514 such that the wire 4502 can be wrapped around the spool 4504. In some configurations, the spool mechanism 4500 can include a spring configured to provide a force on the spool 4504 such that the wire 4502 retracts (i.e., wraps around the spool 4504) once the release button 4510 is actuated.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

What is claimed is:

1. A catheter control system, the system comprising:
a guide catheter comprising a distal end and a proximal end, wherein the distal end is configured to be positioned within an artery;
a guide extension catheter positioned within the guide catheter and configured to extend from the distal end of the guide catheter; and
a catheter control center at a proximal end of the guide catheter, the catheter control center comprising:
a guide extension advancement mechanism, wherein the guide extension advancement mechanism is in communication with the guide extension catheter and is configured to move the guide extension catheter within the guide catheter and extend the guide extension catheter from the distal end of the guide catheter; and
a valve at a proximal end of the catheter control center configured to allow passage of wires and/or devices between the valve and the guide catheter.

2. The system of claim 1, wherein the guide extension catheter comprises a distal end that comprises a tube and a proximal end that comprises a guide extension catheter wire.

3. The system of any of claims 1-2, wherein the catheter control center comprises multiple compartments, wherein the multiple compartments comprise:
a guide extension catheter wire compartment configured to store the guide extension catheter wire; and
a main valve compartment configured to allow passage of wires and devices between the valve and the guide catheter.

4. The system of any of the preceding claims, wherein the guide extension advancement mechanism is configured to deliver equipment through the guide extension catheter.

5. The system of any of the preceding claims, wherein the guide catheter comprises a rail or channel that the guide extension catheter moves along within the guide catheter.

6. The system of any of the preceding claims, wherein the guide extension advancement mechanism comprises a branched valve comprising a first channel and a second channel, wherein the first channel is configured to deliver equipment and the second channel is configured to allow the guide extension catheter and/or the guide extension catheter wire to pass.

7. The system of claim 6, wherein the guide extension catheter wire is in communication with the guide extension catheter and the guide extension catheter moves in coordination with the guide extension catheter wire.

8. The system of any of claims 6-7, wherein the guide extension catheter wire is in communication with the first channel.

9. The system of any of claims 6-8, wherein the guide extension catheter wire is in communication with the second channel.

10. The system of any of claims 6-9, wherein the guide extension advancement mechanism is configured to move the guide extension catheter wire and/or the guide extension catheter within the guide catheter.

11. The system of any of the preceding claims, wherein the guide extension advancement mechanism comprises a slider mechanism.

12. The system of any of the preceding claims, wherein the guide extension advancement mechanism comprises a spool mechanism.

13. The system of any of the preceding claims, wherein the guide extension advancement mechanism comprises a contact wheel mechanism.

14. The system of any of the preceding claims, wherein the guide extension advancement mechanism comprises a screw mechanism.

15. The system of any of the preceding claims, wherein the guide extension advancement mechanism comprises a rack and pinion mechanism.

16. The system of any of the preceding claims, wherein the guide extension advancement mechanism comprises a non-contact mechanism.

17. The system of any of the preceding claims, wherein the guide catheter is configured to be placed into arteries for cardiological or vascular procedures.

18. A catheter control system comprising one or more of the features of the foregoing description.

19. A method of using the catheter control system comprising one or more features of the foregoing description.
